(19) United States
(12) Patent Application Publication
(10) Pub. No.: US 2025/0186406 A1
(43) Pub. Date: Jun. 12, 2025

(54) OPTIMIZING IMMUNOSUPPRESSION IN TRANSPLANT PATIENTS USING PERSONALIZED PHENOTYPIC DOSING MODEL

(71) Applicants: UNIVERSITY OF FLORIDA RESEARCH FOUNDATION, INCORPORATED, Gainesville, FL (US); THE REGENTS OF THE UNIVERSITY OF CALIFORNIA, San Diego, CA (US)

(72) Inventors: Ali ZARRINPAR, Gainesville, FL (US); Chih-Ming HO, San Diego, CA (US); Julia BRUNER, Gainesville, FL (US); Sergio DUARTE, Gainesville, FL (US); Curtis WARREN, Gainesville, FL (US); Duncan LEWIS, Gainesville, FL (US); Neeraj PADMANABHAN, San Diego, CA (US); Raja AL-BAHOU, Gainesville, FL (US)

(21) Appl. No.: 18/704,191

(22) PCT Filed: Oct. 28, 2022
(86) PCT No.: PCT/US2022/048225

§ 371 Date: Apr. 24, 2024

Related U.S. Application Data
(60) Provisional application No. 63/346,019, filed on May 26, 2022.
(60) Provisional application No. 63/273,820, filed on Oct. 29, 2021.

(51) Int. Cl. A61K 31/436; A61K 31/365; A61K 31/5377; A61K 31/573; A61K 39/395; A61P 37/06

(52) U.S. Cl. CPC A61K 31/436; A61K 31/365; A61K 31/5377; A61K 31/573; A61K 39/3955; A61P 37/06

(57) ABSTRACT

The art does not provide systematic and reproducible methods to personalize dosing of multiple immunosuppressive medications after transplantation. This invention provides a method to systematize multi-drug immuno suppression management in tissue and organ transplantation using an artificial intelligence-based complex systems approach. In embodiments of this invention, immunosuppression drug dose, blood drug concentrations, donor-derived fraction of cell free DNA (dd-cfDNA %), and aspartate aminotransferase are used to indicate allograft status or a proxy for allograft status, to generate a phenotypic response surface to produce individual treatment modalities and dosages using empirically determined unique coefficients. This surface is used to calculate appropriate immunosuppression drug doses associated with the desired outcome for that patient. Embodiments of this disclosure are directed to identifying optimized combinations of inputs for the complex system of the immunosuppressed transplant patient in order to avoid transplant rejection while avoiding unnecessary toxicity and maintaining a robust enough immune response to fight infection.

### CROSS-REFERENCE TO RELATED APPLICATIONS

This application is a PCT International Patent Application which claims the benefit of U.S. provisional application Ser. No. 63/273,820, filed 29 Nov. 2021 and U.S. provisional application Ser. No. 63/346,019, filed 26 Mar. 2022. The entire contents of each of these applications are hereby incorporated by reference as if fully set forth herein.

### BACKGROUND

### 1. Field of the Invention

The invention relates to the field of medicine, and in particular to transplant medicine. Embodiments of the invention provide a method that can apply phenotypic personalized medicine (PPM) to immunosuppression dosing in tissue and organ transplant recipients to assess the function of the transplanted material and the immune system of the patient.

### 2. Background of the Invention

Immunosuppression in transplant patients prevents acute rejection and improves both graft and patient survival after transplantation. However, conventional immunosuppression dosing practices rely on data from population-based trials. Such research compiles data from large numbers of individuals and establishes dosing protocols using composite findings. This places patients at risk for infection and toxicity. Immunosuppression management is further complicated by individual variability in drug dosing, metabolism, and immune response. Hence adjusting immunosuppression for any particular patient continues to be a clinical challenge. This existing dosing strategy fails to account for significant inter- and intra-patient variability, frequently resulting in over- or underimmunosuppression. Therefore, there is a great need in the art for methods of controlling transplant immunosuppression in individual patients.

Current drug discovery efforts have primarily focused on identifying agents that tackle specific preselected cellular targets. However, in many cases, a single drug does not correct all of the functioning pathways that affect immunosuppression to produce an effective treatment or immunity moderator. Drugs directed at an individual target often have limited efficacy and poor safety profiles due to various factors, including compensatory changes in cellular networks upon drug stimulation, redundancy, crosstalk, and off-target activities. The use of drug combinations that act on multiple targets has been shown to be a more effective treatment strategy. This is important in controlling immunosuppression such that the transplanted tissue or organ is not rejected, while maintaining sufficient immunity for the patient to avoid opportunistic infections that endanger patient health.

However, while a drug combination can be effective, developing optimized drug combinations for clinical trials can be extremely challenging. For example, even a small number of different drugs (six drugs) each tested at a few concentrations (seven dosages) results in 7⁶=117,649 combinations. Screening all 117,649 combinations through in vitro tests for the most desirable combination is an enormous task in terms of labor and time. Also, a drug combination being effective in vitro does not always indicate that the same drug combination would be effective in vivo. Traditionally, when a drug combination is successfully validated in vitro, the combination is applied in vivo, either by keeping the same dosage ratios or by adjusting the drug administration to achieve the same drug blood levels as attained in vitro. This approach can suffer from absorption, distribution, metabolism, and excretion (ADME) issues. ADME describes the disposition of a pharmaceutical compound within an organism, and the four characteristics of ADME can influence the drug levels, kinetics, and, therefore, efficacy of a drug combination. The discontinuity from cell line to animal and from animal to human as a result of different ADME poses a major barrier to efficiently identifying optimized drug combinations for clinical trials.

Variability in response to immunosuppression results in part due to differences in drug metabolism. Immunosuppressive drugs are substrates of cytochrome P450 and P-glycoprotein, both with genetically variable expression levels in the intestine and liver. The metabolism and clearance of most immunosuppressants are highly dependent on liver and kidney function, both of which can vary tremendously in the post-transplant setting. Other sources of variability in metabolism include physiologic, epigenetic, and environmental factors and site-specific practices. In addition, there is a large and unknown variability in factors mediating immunophenotypes in response to immunosuppression. The complexity of how any individual patient's immune system is altered by a given dose/drug combination is further complicated by the fact that there is no simple measure of how immunosuppressed that individual may be.

Accounting for patient variability is important for minimizing the serious side-effects and toxicities of immunosuppressive agents while maintaining their desired effects. Despite their efficacy in preventing rejection and improving outcomes, immunosuppressive drugs have narrow therapeutic ranges and serious risks, including increased rates of cardiovascular events, malignancies, neurotoxicity, and infections. For example, calcineurin inhibitors (CNIs) are nephrotoxic. Moreover, gains from lower incidence of acute rejection from aggressive immunosuppression are often counterbalanced by negative effects leading to worse death-censored graft survival from infections or cancers. Conversely, underdosing puts patients at risk of acute rejection and accumulation of subclinical allograft damage.

Considering the need to balance these risks, current population-based dosing strategies are inadequate. The idealized patient, as identified by population-based trials, is rarely representative of individual phenotypes. A dosing approach that integrates patient variability could improve patient outcomes while minimizing adverse effects.

### SUMMARY OF THE INVENTION

Therefore, there is a need in the art for a method to determine the optimal combination of drugs to achieve the goal of preventing tissue or organ graft rejection while still maintaining immunity in the patient against pathogens.

This invention concerns a method to systematize multi-drug immunosuppression dosing using an artificial intelligence-based complex systems approach called phenotypic personalized medicine (PPM). PPM applies nonlinear regression to an individual patient's phenotypic dose-response data to generate a mathematical representation of their response to immunosuppressive agents. This representation, termed Phenotypic Response Surface (PRS), allows for the identification of a drug/dose combination likely to produce the desired clinical outcome.

The phenotype chosen to optimize in this study was donor-derived cell-free DNA (dd-cfDNA) fraction, a surrogate biomarker of allograft injury after kidney transplantation. Cell-free DNA are non-encapsulated DNA fragments released into the bloodstream upon cell death and turnover. After organ transplantation, total cfDNA in circulation includes cfDNA released from donor tissues. A greater fraction of dd-cfDNA indicates allograft dysfunction and has been associated with renal allograft rejection in multiple studies. PPM relates individual dd-cfDNA phenotype to treatment, allowing for greater specificity and sensitivity in immunosuppression prescription.

Embodiments of this invention use an AI-based computational drug-dose optimization platform (phenotypic personalized medicine (PPM)) to optimize posttransplant immunosuppression in a subject. Using empiric measurements of allograft injury as quantified by dd-cfDNA and correlating these with a patient's response to the immunosuppression regimen, the method according to embodiments of the invention allows the practitioner to optimize the treatment and suggests subsequent doses in order to minimize graft injury and maximize patient health.

The present invention has demonstrated, in a retrospective cohort, that optimizing multi-input systems can be applied to multi-drug immunosuppression after transplantation. In particular, the invention relates to a method of treating a subject in need of immunosuppression, comprising: (a) administering a combination of N immunosuppressive drugs to the subject; (b) performing a time course of p measurement instances of the dosages of the N drugs in the patient; (c) performing a time course of p measurement instances of the therapeutic outcome of the patient in response to the N immunosuppressive drugs; (d) fitting results of the measurements of the dosages of the N drugs in the patients and the therapeutic outcomes of the patients into a model of the therapeutic outcome; (e) using the model of the therapeutic outcome to identify optimized dosages of the N immunosuppressive drugs; and (f) treating the patient with the optimized dosages of the N immunosuppressive drugs, wherein N is an integer of 2 or more, wherein the model of therapeutic outcome is a quadratic function of dosages of the N drugs with m parameters where m=1+2N+(N(N−1))/2; wherein m is the number of parameters needed, and wherein p≥m.

Preferably, the subject is a transplant patient who has received a transplant of tissue or one or more organs, for example one or more of bone marrow, liver, kidney, heart, lung, pancreas, intestine, skin, and a combination thereof.

In certain embodiments, N is 3 or more. In other embodiments, p=m.

In certain embodiments, the N immunosuppressive drugs include at least one calcineurin inhibitor and at least one glucocorticoid. In certain embodiments, the N immunosuppressive drugs are selected from two or more of the group consisting of tacrolimus, sirolimus, everolimus, zotarolimus, cyclosporine, dactinomycin, methotrexate, prednisone, mycophenolate motetil, azathioprine, basilixirab, cyclophosphamide, mycophenolic acid, and Methylprednisolone.

In particular embodiments, the present invention relates to a method of providing immunosuppressive therapy to a subject in need of immunosuppression, comprising: a) administering known dosages of a combination of N immunosuppressive drugs to the subject; b) performing a time course of p measurement instances of the dosages of the N drugs in the patient; c) performing a time course of p measurement instances of the therapeutic outcome of the patient in response to the N immunosuppressive drugs; d) fitting results of the measurements of the dosages of the N drugs in the patients and the therapeutic outcomes of the patients to a prediction of therapeutic outcome; e) using the model of the therapeutic outcome to identify preferred changes to the dosages of the N immunosuppressive drugs; and f) treating the patient with the changed dosages of the N immunosuppressive drugs, wherein N is an integer of 2 or more, wherein the model of therapeutic outcome is a quadratic function of dosages of the N drugs with m parameters where m=1+2N+(N(N−1))/2, wherein m is the number of parameters needed, and wherein p≥m.

In certain embodiments, the invention relates to a method wherein the subject is a transplant patient who has received a transplant of tissue or one or more organs, preferably wherein the tissue or organ is selected from the group consisting of bone marrow, liver, kidney, heart, lung, pancreas, intestine, skin, and a combination thereof.

In certain embodiments, the invention relates to methods wherein N is 3 or more. In certain embodiments, the invention relates to methods wherein p=m. In certain embodiments, the invention relates to methods wherein the N immunosuppressive drugs include at least one calcineurin inhibitor and at least one glucocorticoid.

In certain embodiments, the inventive methods are those wherein the N immunosuppressive drugs are selected from two or more of the group consisting of tacrolimus, sirolimus, everolimus, zotarolimus, cyclosporine, dactinomycin, methotrexate, prednisone, mycophenolate motetil, azathioprine, basiliximab, cyclophosphamide, mycophenolic acid, and methylprednisolone.

In certain embodiments, the invention relates to methods wherein p comprises immunosuppression drug dose, blood drug concentrations, donor-derived fraction of cell free DNA (dd-cfDNA %), or aspartate aminotransferase.

### BRIEF SUMMARY OF THE DRAWINGS

Certain embodiments are illustrated by way of example, and not by way of limitation, in the figures of the accompanying drawings.

FIG. 1 is a flow chart schematic showing an example of PPM methods.

FIG. 2 is a flow chart showing an example of some of the steps of inventive methods.

FIG. 3A, FIG. 3B, and FIG. 3C are a set of graphs outlining the cycles of input and output in a generic example method.

FIG. 4 is a flow chart showing the methods for the PRS equation.

FIG. 5 is a graph showing data from a pilot clinical trial of PPM dosing versus control.

FIG. 6A and FIG. 6B are a set of graphs showing data from a prospective clinical trial of PPM dosing versus control. The data show the ratio of days of more than 2 ng/mL outside the target range (FIG. 6A) and post-transplant length of stay (LOS) (FIG. 6B).

FIG. 7A, FIG. 7B, and FIG. 7C are a set of graphs showing multidrug immunosuppression PPM dosing in kidney transplantation. FIG. 7A: PPM dosing consistently recommended increasing immunosuppression in a patient who ultimately suffered from acute rejection. Conversely, PPM dosing recommended decreasing immunosuppression in patients who later had recurrent urinary tract infections (FIG. 7B) and ureteral strictures leading to elevated creatinine levels (FIG. 7C). Circle: actual dose; Star: PPM dose. MMF indicates mycophenolate mofetil.

FIG. 8A is a flow chart showing the underlying pathophysiology of chronic calcineurin toxicity. FIG. 8B is a flow chart showing the therapeutic potential of dd-cfDNA monitoring of allograft injury with optimized immunosuppression dosing. FIG. 8C is a conceptual model of the basis of the invention.

FIG. 9A and FIG. 9B are tables showing the clinical trial schedule for the indicated weeks.

FIG. 10A and FIG. 10B are graphs showing the changes in tacrolimus and prednisone 14 days before rejection, t day before rejection, and 40 days after rejection in two separate patients. FIG. 10A is Patient 1; FIG. 10B is Patient 2.

FIG. 11 shows the PPM recommended ΔIS.

FIG. 12 is a table showing patient demographics.

FIG. 13 is a boxplot of PPM recommended changes in immunosuppression according to recommendation class.

FIG. 14 shows a boxplot of PPM recommended changes in immunosuppression according to recommendation class.

FIG. 15 show graphs depicting stringent PPM recommended ΔIS and PPM method comparison.

FIG. 16 show graphs comparing AlloSure and AlloMap.

FIG. 17 shows recommended changes in immunosuppression.

FIG. 18 is a scatter plot of patients' measured dd-cfDNA fraction versus the dd-cfDNA fraction projected by PRS functions at the physicial-prescribed dosing combination.

FIG. 19 shows the recommended change in immunosuppression guided by dd-cfDNA fraction (AlloSure) versus gene expression analysis score (AlloMap).

### DETAILED DESCRIPTION OF EMBODIMENTS OF THE INVENTION

### 1. Overview

An artificial intelligence systems approach, using phenotypic personalized medicine (PPM), was used to determine individualized responses to treatment for multi-drug immunosuppression in kidney transplantation. PPM uses immunosuppression drug dose, blood drug concentrations, and donor-derived fraction of cell free DNA as an indication of allograft status, to generate a treatment response surface. This surface is then optimized to give the desired immunosuppression drug response via dosing of the drugs.

In general, response surface methodology explores the relationships between several explanatory variables and one or more response variables. The main idea is to use a sequence of designed experiments to obtain an optimal response using a polynomial model. The Phenotypic Response Surface (PRS) function (Function 1) was obtained by inductive approach from experimental evidence and is a quantitative input-output transfer function of biological complex system. The PRS function can be employed to maximize the desired results or features of a process by optimization of operational factors. In contrast to conventional methods, the interaction among process variables can be determined by statistical techniques.

These methods allow single or multi-objective parameters (e.g., efficacy, safety, optimal dosage, and other parameters) to be considered during the course of the treatment. In particular, dosages of immunosuppressive drugs can be administered in the ideal combination and dose for an individual subject which provide sufficient immunosuppression to retard or eliminate rejection of an implant while still retaining as robust an immune system in the patient as possible, to avoid frequent and serious infection. The method also dramatically reduces the time necessary for trial-and-error testing for the patient, as well as the number and cost of tests, based on a response of the system to designed time-varying stimulations, optimized combinatorial stimulations can be identified in a reduced or minimal number of test cycles, even down to one test cycle. Further, the method allows identification of an optimized, time-varying treatment for individual subjects during immunosuppressive transplant treatments is over period of time. The method does not rely on the availability of detailed information for the complex system under control and allows the practitioner to optimize time intervals between applying various stimulations can be identified.

### 2. Definitions

Unless defined otherwise, all technical and scientific terms used herein have the same meaning as commonly understood by one of ordinary skill in the art. Although various methods and materials similar or equivalent to those described herein can be used in the practice or testing of the present invention, suitable methods and materials are described below. However, the skilled artisan understands that the methods and materials used and described are examples and may not be the only ones suitable for use in the invention. Moreover, as measurements are subject to inherent variability, any temperature, weight, volume, time interval, pH, salinity, molarity or molality, range, concentration and any other measurements, quantities or numerical expressions given herein are intended to be approximate and not exact or critical figures unless expressly stated to the contrary.

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, element or step or group of items, elements or steps. Furthermore, the indefinite article “a” or “an” is meant to indicate one or more of the item, element or step modified by the article.

As used herein, the term “about” means plus or minus 20 percent of the recited value, so that, for example, “about 0.125” means 0.125±0.025, and “about 1.0” means 1.0±0.2. Notwithstanding that the numerical ranges and parameters setting forth the broad scope of the invention are approximations, the numerical values set forth in specific non-limiting examples are reported as precisely as possible. Any numerical value, however, inherently contains certain errors necessarily resulting from the standard deviation found in their respective testing measurements at the time of this writing. Furthermore, unless otherwise clear from the context, a numerical value presented herein has an implied precision given by the least significant digit. Moreover, all ranges disclosed herein are to be understood to encompass any and all sub-ranges subsumed therein. For example, a range of “less than 10” can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

As used herein, the term “treatment response surface” refers to the multidimensional surface representing the changes in the quantifiable outcome that results from to modulations in treatments. The phrase “phenotypic response surface: refers more broadly to the multidimensional surface representing the changes in the quantifiable observable characteristic of interest that result from changes in any variables affecting that individual or system.

As used herein, the term “measurement instance” refers to individual and independent measurements of a quantifiable observable characteristic.

As used herein, the term “immunosuppression” is the partial or complete suppression of the immune response of an individual, either naturally as a result of disease or another condition or artificially induced to help the survival of an organ after a transplant operation.

As used herein, the term “immunosuppressive drug” refers to a class of drugs that suppress or reduce the strength of the body's immune system.

As used herein, the term “personalized phenotypic medicine (PPM)” refers to an approach that finds an appropriate drug combination using a quadratic phenotypic optimization platform and an appropriate dosing strategy over time based on small data collected exclusively from the treated individual.

As used herein, the term “stimulations” refers to alterations in the system by inputs.

As used herein, the term “donor-derived cell-free DNA” refers to the cell-free (non-encapsulated) DNA derived from apoptosis or necrosis of allograft tissue, which circulates in the body fluids of patients after organ transplantation. This measurement is a proxy for the health of the donor tissue.

As used herein, the term “calcineurin inhibitor” refers to a group of compounds that inhibit calcineurin, a T cell activator, causing a reduction in T cell activity. Examples include tacrolimus and/or cyclosporine.

As used herein, the term “glucocorticoid” refers to any of a group of corticosteroid steroid hormones that reduce inflammation. Examples include hydrocortisone, cortisone, prednisone, prednisolone, methylprednisolone, dexamethasone, triamcinolone, fludrocortisone, or betamethasone.

As used herein, the terms “aspartate aminotransferase”, “aspartate transaminase” or “AST” refer to an important enzyme in amino acid metabolism. AST is a pyridoxal phosphate (PLP)-dependent transaminase enzyme that catalyzes the reversible transfer of an α-amino group between aspartate. AST is found in the liver, heart, skeletal muscle, kidneys, brain, red blood cells, and gall bladder.

### 3. Summary of Results

This invention provides a systematic multi-drug personalization method using an artificial intelligence-based complex systems approach called Phenotypic Personalized Medicine (PPM). PPM relies on empiric data, in this case immunosuppression drug dose, blood drug concentration, and donor-derived cell-free DNA fraction, to generate time-dependent personalized treatment response surfaces. This surface is then used to identify optimal immunosuppression drug doses associated with the desired outcome. In a retrospective analysis of 6 (is this the correct number) kidney transplant patients, dose adjustment recommendations were made at 17 distinct timepoints. PPM was able to distinguish between clinically stable, future infection, and future rejection episodes and recommend appropriate immunosuppression adjustments accordingly. PPM recommended increased immunosuppression at 9/11 (81.8%) timepoints prior to rejection episodes and decreased immunosuppression at 7/8 (87.5%) timepoints prior to infection episodes. For stable patients, PPM recommendations were divided: 15 to increase and 14 to decrease. Initial investigation in this cohort suggests PPM can assist clinicians in systematically addressing variability in patient drug responses to improve post-transplant multi-drug immunosuppression management.

### 4. Embodiments of the Invention

### Discussion

This invention provides a method to systematize multi-drug immunosuppression management in kidney transplantation using an artificial intelligence-based complex systems approach called phenotypic personalized medicine (PPM). PPM relies on clinical data. In summary, embodiments of the invention include an artificial intelligence-based immunosuppression optimization in transplantation.

A further benefit of the disclosed technique is that drug dosages can be individually tailored for a subject based on phenotypic responses of the subject to realize phenotypic personalized medicine, since individually optimized drug dosages for one subject can differ a great deal from those individually optimized for another subject. By adjusting or tuning drug dosages (or drug dosage ratios) according to individual phenotypes, an individually optimized drug-dosage combination can be designed to accommodate an individual's own immune system.

For embodiments of this invention, immunosuppression drug dose, blood drug concentrations, and donor-derived fraction of cell free DNA (dd-cfDNA %) is used as an indication of allograft status or a proxy for allograft status, to generate individual treatment response surfaces. Each patient's phenotypic response surface (PRS) is empirically determined and has unique coefficients. Once generated, this surface is used to calculate appropriate immunosuppression drug doses associated with the desired outcome for that patient. In a retrospective analysis of 8 kidney transplant patients, PPM recommended increased immunosuppression in 3 of 3 patients who later went on to experience biopsy-confirmed rejection. In the 2 patients with suspected rejection but without biopsy confirmation, PPM recommendations were split: in one patient recommending decreased immunosuppression and in the other recommending increased immunosuppression. PPM also identified the need for decreased immunosuppression in 2 of 2 patients who later developed infections. In the single analyzed case of stable allograft status, PPM recommended a modest increase in immunosuppression. Thus, a retrospective study of systematic AI-based individualized immunosuppression dosing has demonstrated an actionable strategy for multi-drug immunosuppression management.

The use of calcineurin inhibitors (CNIs) to prevent or reduce transplant rejection is a mainstay of treatment after transplantation, but it is associated with nephrotoxicity. The introduction of CNIs like tacrolimus and cyclosporine has greatly reduced the incidence of acute rejection and improved graft and patient survival after transplantation. However, these drugs have narrow therapeutic ranges and serious side-effects and toxicities, including increased rates of cardiovascular events, malignancies, neurotoxicity, and infections. Nephrotoxicity in particular is a major problem in kidney transplantation, as the kidney is particularly vulnerable in this setting. Even in non-kidney transplant patients or patients with other indications for CNIs, patients can have an up to 45 percent reduction in glomerular filtration rates (GFRs) compared to those not on CNIs.

Chronic CNI nephrotoxicity leads to renal insufficiency from vasculopathy, glomerular disease, abnormal tubular function, and hypertension. Biopsies consistently show primary endothelial damage as manifested by obliterative arteriolopathy. There is also glomerular scarring or collapse, tubular vacuolization, glomerulosclerosis, tubular atrophy, and interstitial fibrosis. Clinically, it presents as a progressive decline in renal function with increasing proteinuria and hypertension. This irreversible process appears to be due to the combined effects of hemodynamic changes and direct toxicity on renal tubular epithelial cells. Accompanying these effects are thrombotic microangiopathy and electrolyte disturbances.

In the last decade, many studies have tested strategies to minimize CNI exposure in kidney transplantation. Apart from their unfavorable risk profile, both tacrolimus and cyclosporine are inherently nephrotoxic and associated with premature graft loss. Reduction of CNIs has been shown in some studies to improve graft survival. However, under-immunosuppression leads to alloimmune reactions of either acute or chronic rejection, leading also to graft failure and loss. Minimizing the immunosuppression or using alternative modalities for immunosuppression can be used as strategies to reduce CNI toxicity. Complete cessation or replacement of CNIs by mTOR-Is (a mammalian target of rapamycin inhibitors) such as everolimus in de novo transplant recipients has resulted in unacceptably high early acute rejection rates even with induction antibody therapy. A Cochrane review of 83 such studies has found that CNI minimization/withdrawal results in more short term acute rejection episodes, but no significant change in graft or patient survival or cancer or infection risks.

Multiple strategies have been attempted to alleviate these problems, including i) CNI withdrawal, ii) low dose CNI, iii) CNI withdrawal with conversion to mTOR-Is, and iv) low dose CNI with addition of mTOR-I. Few if any of these strategies have been determined to actually improve kidney function or allograft survival. Taken as a whole or individually, none of these strategies changed outcomes significantly. In addition, there have been no long term follow-up to determine the results of these strategies. In summary, CNIs are the baseline immunosuppressant drugs in transplant patients, but they have toxicities which have been intractable.

The toxicities and side effects of CNIs are not the only problem. Over- or under-immunosuppression overall is also a major difficulty for transplant patients. Any gains from lower incidence of acute rejection by using aggressive immunosuppression are often counterbalanced by negative effects leading to worse death-censored graft survival from infections or cancers. In fact, there has not been much of an increase in long-term graft survival despite great improvements in early patient and graft outcomes due to surgical and perioperative management of transplant recipients. Many clinicians have argued that an individualized immunosuppressive strategy is needed with specific attention to patient variability in the factors of CNI toxicity, infection, and rejection. Inter- and intra-individual variability in dosing requirements, particularly across different patient populations, necessitates empirical physician-titrated drug administration that frequently results in deviation from target ranges and over- or under-immunosuppression. While short-term and medium-term outcomes with solid organ transplantation has greatly improved over the last several decades, largely due to better immunosuppression management, optimal long-term maintenance therapy after transplantation remains to be defined. It is likely to be a dynamic, as opposed to a stable process.

In some embodiments, an outcome of a complex system in response to stimulations can be sensitive to temporal features (i.e., the timing of close administration) as well as the dose amount itself. Thus, the drugs administered, the doses of those drugs, and the timing of those dose administrations can be tested in a subject for the corresponding dynamically changing outcome in that subject, and the experimental results of the tests are then fitted into a model of the system, such as by using multi-dimensional fitting. Based, on analysis of the correlation between the modulated features of the treatment modality and the patient outcome, one can identify the optimized combinations of these features so that the patient can receive personally optimized treatment.

Thus, the significant degree of patient variability requires a strategy that can individualize treatment. Post-transplant immunosuppression provides a challenging model to test any precision medicine platform. Previous studies have sought to personalize immunosuppression dosing using genetics, population pharmacokinetics, and other predictive modeling approaches. However, it is difficult to simultaneously account for inter- and intra-individual variability in treatment needs, not to mention center-specific practices. These differences also lead to wide racial health disparities that are not solely attributable to access, economics, or adherence, even when while maintaining excellent control of calcineurin levels. Immunosuppressive drugs are substrates of cytochrome P450 and P-glycoprotein (also known as MDR1 and ABCB1), both with genetically variable expression levels in the intestine and liver. The metabolism and clearance of most immunosuppressants are highly dependent on liver and kidney function, both of which can vary tremendously in the post-transplant setting. Furthermore, all patients are on multiple interacting medications. A simple algorithm will not be able to respond adequately to this complexity.

Precision medicine-based noninvasive measures of graft injury as used in embodiments of the inventive methods described here include the measurement of donor-derived cell-free DNA (dd-cfDNA) in the plasma of transplant recipients. As sequencing and computational techniques have improved, so has the ability to detect and differentiate recipient cfDNA from that of the donor. Clinical studies from multiple centers have shown that dd-cfDNA quantities in the recipient plasma can distinguish allograft injury. A fraction of dd-cfDNA greater than 1% indicates graft injury from active rejection or infection. This method requires only a blood draw, as opposed to the more complex and labor-intensive histologic analysis of an allograft biopsy, and can therefore be used more easily to monitor allograft status. Therefore, tracking dd-cfDNA content is used here as a marker of graft status.

Phenotypic Personalized Medicine (PPM) is used here to mediate mechanism-independent and patient-specific optimization of immunosuppression. PPM is a platform to systematically search for optimal treatment combinations. The methods here involve a powerful platform that utilizes patient clinical data to construct a Phenotypic Response Surface (PRS). The PRS is patient-specific and based on individualized constants that represent each individual patient's response to drug treatment. Examples of this response optionally include tacrolimus blood trough levels and quantitative markers of organ function or injury. PRS reconciles clinical data into a visual map that enables the immediate identification of optimal drug doses needed to bring drug levels to within the best range in terms of function in the individual patient. Importantly, because the PRS process does not require any a priori knowledge of disease mechanism, it can efficiently prescribe precise and optimized drug doses despite the frequent changes to patient treatment regimens following transplantation that can have a profound effect on drug metabolism.

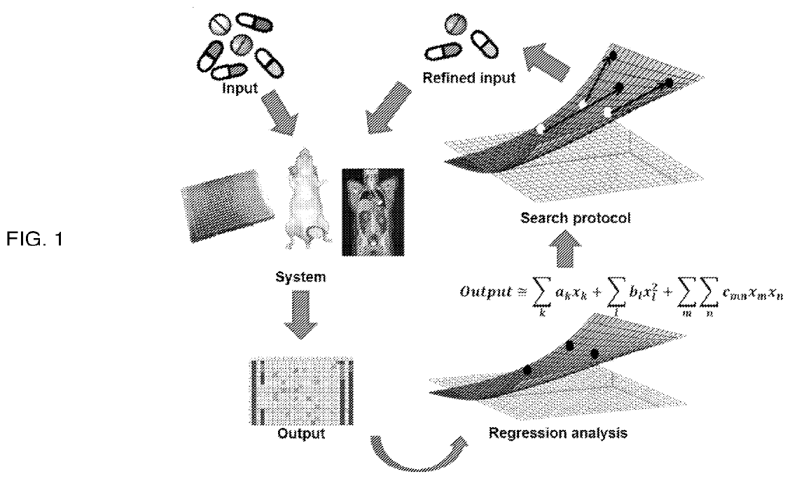

See FIG. 1, a schematic showing the general components of the PPM optimization process as used in embodiments of the invention to pinpoint a globally optimal outcome of the therapy. FIG. 1 first shows at the upper left, (a) the input, drug combinations with defined doses, then (b) the system, the disease or process to be studied, (c) the output, the quantitative response to the input, (d) the statistical regression analysis, used to analyze input-output relationships to guide optimization, and (e) the search protocol, which drives the system output toward the desired response and (f) refined input.

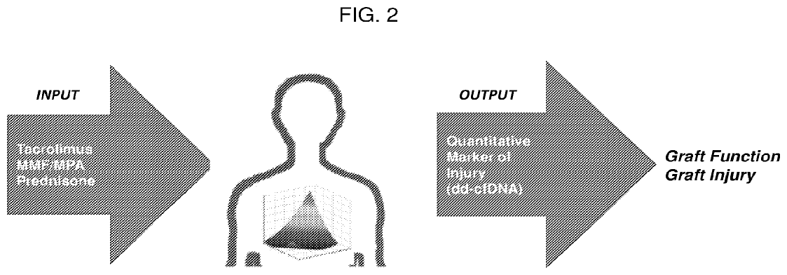

Here, PPM dosing combined with dd-cfDNA in the inventive methods surprisingly far exceeds the information and the decision-making potential of prior methods using creatinine levels and physician-guided dosing. Because PPM implicitly incorporates mechanistic information about disease processes, including genetic variability, environmental contributions, and physiologic variations, these factors will not need to be explicitly known or used. Instead, only the variables that change over the timescale of the study, i.e. the drugs being administered to the patient, are used as inputs for the calculation. See FIG. 2, a flow chart showing an example of the method.

The method here implicitly incorporates mechanistic information such as genomics, proteomics, pharmacokinetics, and other components of disease biology, and takes into consideration the genetics, patient environment (i.e., other medications, etc.), patient heterogeneity, epigenetics, and pharmacokinetics. Explicit information pertaining to these factors therefore is not necessary to optimize personalized combination therapy regimens for immunosuppression. The primary components of the method here are the inputs, comprising the therapies, and the outputs, which are quantifiable indicators of treatment efficacy and safety. Thus, combining dd-cfDNA and an AI-based combination drug-dose optimization improves the clinical outcome in any population. Allograft injury surveillance using embodiments of the inventive methods allows the practitioner to decrease graft injury and improve graft function and histology.

The large array of genetic, environmental, and physiologic factors that affect each individual's drug response makes generating a predictive algorithm for the selection of immunosuppression drug combinations and doses very difficult. As such, there is a clear need to minimize calcineurin toxicity and personalize post-transplant drug treatment. This need is a way to combine a way to monitor each patient's immunosuppression state and to personalize post-transplant drug regimens accordingly.

In summary, the invention allows a mechanism-independent and patient-specific optimization of immunosuppression, preferably using clinical data such as tacrolimus concentration or blood trough levels and/or quantitative markers of organ function or injury (e.g., dd-cfDNA). These are individualized constants that represent each patient's response to drug treatment. The PRS reconciles the clinical data into a visual map that enables the immediate identification of drug doses needed to achieve optimal results. This method can efficiently identify precise and optimized drug doses despite frequent changes to the treatment regimens following transplantation that can have a profound effect on drug metabolism and response. Through case-specific drug design, the design of drug combinations can compromise and balance between different or opposing patient outcome criteria, thereby identifying optimal drug combinations on a patient-by-patient basis.

In general, the methods of the invention, in some embodiments, relate to a method of optimization of administering courses of transplant immunosuppressive drugs which involves (a) administering the immunosuppressive drugs to a subject (including optionally changing the dosing and/or the drug(s) over time), (b) measuring over time factors such as drug concentration in the subject, the condition of the allograft, the level of immunosuppression, and the like, (c) fitting the time-varying response of the subject to a model of the system; (d) using the model of the system to identify an optimized combination of dosages for that subject to produce a desired level of immunosuppression for the patient that prevents rejection of the allograft without over-immunosuppression that can endanger the patient with respect to infections or unnecessary toxicity.

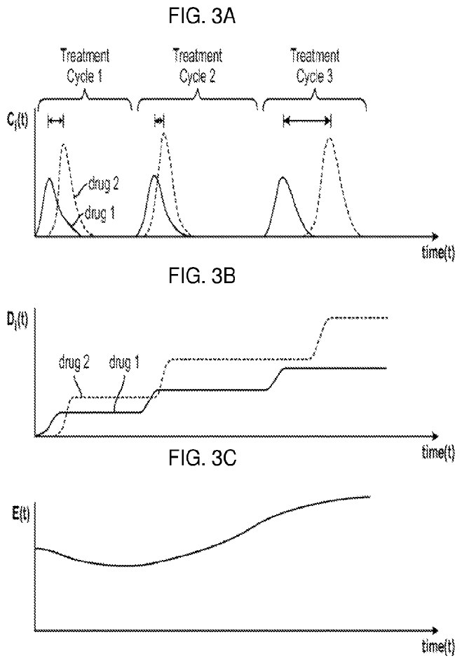

The methods thus can involve cycles of treatment and testing to improve with each cycle. See, for example FIG. 3, which shows a generalized example of treatment cycles with two drugs (FIG. 3A), and the resulting cumulative drug dosages (FIG. 3B), and the therapeutic outcome (FIG. 3C). According to this moving time window approach, for example, the drug dosages applied to the test subject at a next treatment (here, cycle 4) can be optimized at least in part based on measurements performed on the test subject during the immediately preceding treatment cycle 3. Likewise, the drug dosages applied to the test subject at a further treatment (here, cycle 5) can be optimized at least in part based on measurements performed on the test subject during the immediately preceding treatment cycle 4, and so on.

### Subjects

Subjects contemplated for treatments according to embodiments of the invention include any mammal, including humans, laboratory animals, livestock, companion animals, zoo animals, and the like. In particular, the methods according to the invention are contemplated to be useful in humans, apes, monkeys, rats, mice, rabbits, bovines, equines, ovines, caprines, canines, felines, and the like. The preferred subject is a human or a laboratory animal such as rats and mice.

Subjects in need of methods according to embodiments of the invention generally will be experimental animals or humans that have undergone a transplant of tissues or organs and are to be treated with or are being treated with immunosuppressive regimens to retard or prevent rejection of the allograft.

### Organs and Tissues

Transplanted tissues and organs can be any allograft, including solid organs (such as kidney, liver, heart, lungs, pancreas, stomach, intestine, thymus, uterus, testis, ovaries, colon, spleen, parathyroid glands, and the like), tissues (such as bone marrow, bone, cornea, skin, heart valves, nerves, veins, tendons, pancreatic islets, blood, hand, face, skin, beta cells, parathyroid cells, limbs, and the like). Preferred transplants are kidney, heart, lung, intestines, liver, and pancreas.

### Immunosuppressive and Other Drugs

The preferred immunosuppressive drugs contemplated for use with the inventive methods are a calcineurin inhibitor (CI), an antiproliferative, and a glucocorticoid, most preferably tacrolimus, mycophenolate mofetil, and prednisone. However, the invention can be used with any immunosuppressive drug or any combination of immunosuppressive drugs, alone or with additional therapeutic modalities, immunosuppressants usually are divided into classes including calcineurin inhibitors, interleukin inhibitors, selective immunosuppressants and TNF alfa inhibitors. Thus, drugs to be included in the treatment can include any of those, including one or more of tacrolimus, sirolimus, everolimus, zotarolimus, cyclosporine, dactinomycin, methotrexate, prednisone, mycophenolate mofetil, mycophenolic acid, azathioprine, basiliximab, cyclophosphamide, antithymocyte globulin, and belatacept. Preferred immunosuppressants are tacrolimus, prednisone, mycophenolate mofetil.

Glucocorticoids are used with the invention in addition to the immunosuppressants discussed above for immunosuppression treatment. Preferred glucocorticoids include prednisone, methylprednisolone, or hydrocortisone. The most preferred glucocorticoid is prednisone.

The drugs used in combination for immunosuppressive therapy include hydrocortisone, cortisone, prednisone, prednisolone, methylprednisolone, dexamethasone, triamcinolone, fludrocortisone, or betamethasone, however the most common immunosuppressive therapy is a combination of tacrolimus, mycophenolate mofetil, and prednisone. Thus, the invention includes embodiments where any two of these compounds used in transplant immunosuppression therapies are used. Some embodiments of this disclosure provide a method that allows a rapid determination of optimized dosage regimens for treating transplant patients. For example, a combination of 2 or more including 3, 4, 5, 6, 7, 8, 9, 10, or more) drugs can be evaluated to rapidly identify optimized dosages of the drugs.

### Markers and Testing

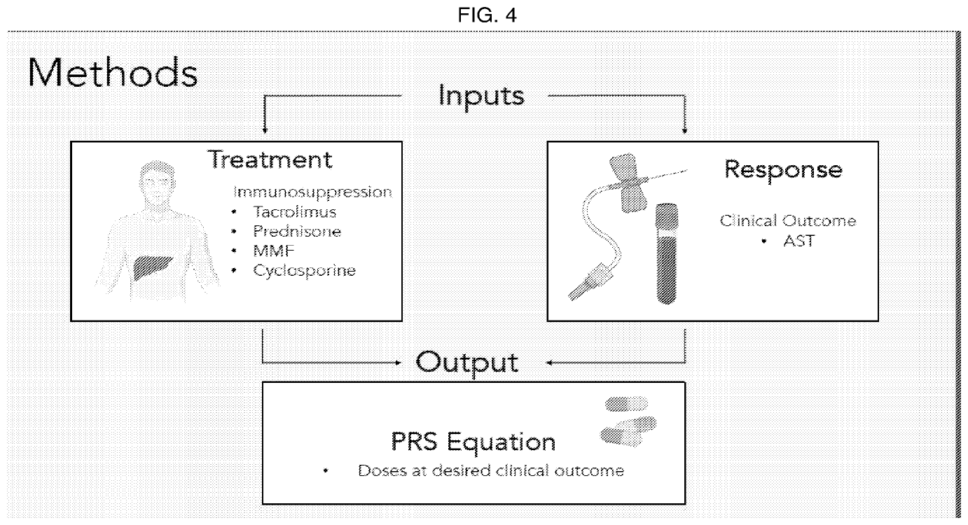

The markers preferably used to assess the condition of the transplanted tissue or organ in preferred embodiments of this invention is donor-derived cell-free DNA (dd-cfDNA) percentage and AST level. Measuring donor derived cell-free DNA (dd-cfDNA) can predict acute rejection or other injury to the transplanted material and serves as an important output measurement. See flow chart in FIG. 2. In general, it is desirable to maintain a dd-cfDNA level of less than 1%. While the lowest dd-cfDNA level is desired, there is a threshold below which the assay is not reliable. Measuring AST level predicts future rejection events and serves as an important input measurement (FIG. 4).

Additional markers of transplant condition that can be used in addition to dd-cfDNA or instead of dd-cfDNA include quantitatable transcriptional values or composite scores (e.g. AlloMap), protein biomarkers (e.g. cytokines), viral counts, other values of organ injury (e.g. alanine aminotransferase, amylase, lipase, troponin).

The cumulative dosages of the CNIs and other drugs delivered to the patient also are measured as an output in embodiments of the invention. For example, tacrolimus and prednisone are administered in known amounts at known times to a subject, after which the calcineurin inhibitor amount in the subject blood is measured. This is a standard-of-care test performed by certified clinical labs using methods such as high-performance liquid chromatography.

Other examples of measurements or outputs of phenotypic responses that can be considered in the inventive methods can include collection of blood, serum, plasma, urine, saliva, sputum, and the like, or other excretions or biological materials such as biopsies for testing. Tests performed on these samples can include tests for cytokines, antibodies, serum proteins, electrolytes, hematocrit levels, other cell counts or cell population characteristics, other biological markers, DNA, RNA, metabolites, or microbiota, and the like. Physical parameters or clinical signs such as patient body temperature, blood pressure, pupil dilation, body weight, fluid intake or excretion, heart rate, urine output, drain output, dialysis volume, can be used with the invention. In addition, imaging techniques, such as X-ray, PET, CT, CAT, MRI (e.g., conventional MRI, functional MRI, or other types of MRI), fluorescence spectroscopy, near-infrared spectroscopy, Raman spectroscopy, fluorescence correlation spectroscopy, acoustic imaging techniques, microscopy of tissue, biopsy, and other imaging techniques to monitor the status of the transplant or to monitor fluid and blood flow to and from the transplant can be used as an indicator of a need to change the immunosuppression level in the patient.

### Models

A model useful for embodiments of the invention here is described in U.S. Pat. No. 10,603,390, which is incorporated herein in its entirety. See columns 6-15. Artificial neural networks were applied to analyze the experimental data of a bio-system, cell, animal or human, response to regimen. The system outcome related to the molecular interactions among drug and disease-causing agents was found to be a simple shaped smooth surface. The phenotypic response surface is represented by a second order polynomial function:

$$E ( C , t ) = {x}_{0} ( {c}_{i} , t ) + {\sum }_{i = 1}^{N} {x}_{i} ( {c}_{i} , t ) {c ( t )}_{i} + {\sum }_{i = 1}^{N} {y}_{ii} ( {c}_{i} , t ) {c ( t )}_{i}^{2} + {\sum }_{i = 1}^{N - 1} {\sum }_{j = i + 1}^{N} {z}_{ij} ( {c}_{i} , t ) {c ( t )}_{i} {c ( t )}_{j} , \qquad Function 1$$

$$E ( C , t ) = {x}_{0} ( {c}_{i} , t ) + {\sum }_{i - 1}^{N} {x}_{i} ( {c}_{i} , t ) {c ( t )}_{i} + {\sum }_{i - 1}^{N} {y}_{ii} ( {c}_{i} , t ) {c ( t )}_{i}^{2} + {\sum }_{i = 1}^{N - 1} {\sum }_{j = i + 1}^{N} {z}_{ij} ( {c}_{i} , t ) {c ( t )}_{i} {c ( t )}_{j} ,$$

where E(C, t) is the outcome, c(t)ᵢ is the dose of different drugs, and x₀, xᵢ, yᵢᵢ, and zᵢᵢ in front of each item are the coefficients. These coefficients are not constant and can be functions other independent parameters and time. Calibration tests are used to find the values of these coefficients. The outcome values can then be determined. The current and the desired outcome values can then be used to determine the changes needed in the treatment to achieve the desired outcome.

Certain phenotypic responses in the case of certain embodiments of this invention, such as prevention of allograft rejection and health of the allograft, are desirable, while other phenotypic responses are undesirable, such as drug toxicity or side effects due to over-immunosuppression. In the case of the latter phenotypic responses, their weighting factors serve as penalty factors in the optimization of the combination of N drugs. Various weighting factors in the algorithm discussed herein and described in U.S. Pat. No. 10,603,390 can be adjusted or tuned to reflect the relative importance of desirable optimization criteria and undesirable optimization criteria, and the adjustment or tuning can be performed on a case-by-case basis to yield different optimized dosages of the drugs to be administered, depending on the particular subject's needs and health parameters. Also, the adjustment or tuning of the weighting factors can be performed over time so as to incorporate feedback over the course of a treatment. This becomes important for transplant patients, who receive immunosuppressive therapy life-long.

A processing unit useful in the invention is described in U.S. Pat. No. 10,603,390, which is incorporated herein in its entirety. See columns 15-16.

### Methods

For treatment of an individual patient, a subject in need is chosen. This subject preferably is a human patient who has received a transplant of tissue, bone marrow, or a solid organ. This patient is initially treated with cycles of immunosuppressive combination drugs, all within the dose ranges approved for use by the United States Federal Drug Agency. As shown in the general scheme in FIG. 3A, treatments are provided, after which the subject is tested for blood drug levels and tests of organ function and injury, and the results are used to construct a new recommendation for treatment modality.

Calculation of each recommendation begins with generation of the phenotypic response surface (PRS). The PRS is a non-linear regression equation which models the patient's reaction to immunosuppressive drug combinations. It is created by applying second order regression to collected clinical data including previously prescribed doses for each drug and a corresponding measurable outcome. Hence, the equation of each model is specific to both the patient and time of recommendation. Optimization of two-drug combinations requires six clinical data points while three-drug optimization requires ten. Once generated, the PRS is used to identify the combination of doses most likely to produce the desired clinical outcome.

In the study exemplified here, the aim was to optimize combinations of tacrolimus, mycophenolate mofetil, and prednisone using the donor derived cell-free DNA fraction as a measure of allograft function. Notably, serum levels of tacrolimus were used rather than the total daily dose, to generate the PRS. Prescribed total daily dose was used for all other IS agents. Before regression, each dose is transformed as a fraction of the maximum. This provides a uniform scale for each dimension of the PRS to reduce error introduced by differences in order of magnitude of each drug's typical dosage. This transformation also useful for quantitation of recommended change in immunosuppression.

Given the consistency of dd-cfDNA and gene expression guided recommendations, the PPM platform shows adaptability to a variety of quantitative phenotypes. However, while dd-cfDNA informed dosing recommendations aligned with clinical practices, we were unable to confirm this trend for doses optimized using transcription scores inputs due to a lack of event related data. Gene expression scores were collected from a smaller subset of patients that did not have enough samples within the defined 14-day window. Hence, a larger cohort of patients will be needed to evaluate the predictive capacity of gene expression scores as PPM inputs in the context of infection and rejection events. Application to a larger cohort will also allow us to confirm dd-cfDNA directed PPM's predictive capacity with greater statistical power.

Despite this, the two phenotypic inputs showed promising consensus in recommendations made for the stable group, which showed the greatest variability in both strength and direction. Provided this considerable variability, agreement across input identities indicates a relatively reliable pattern of recommendation. Based on this initial application and the nature of the PRS function, PPM can demonstrate similar performance using a range of relevant, validated biomarkers.

The physiological effects of immunosuppressants varies significantly among patient phenotypes. As a result, the relationship between conventionally prescribed doses and the overall immunosuppression state is weak. Consequently, traditional population-based and protocol-driven (or more likely individual clinician style-driven) dosing strategies and conventional pharmacokinetics are insufficient, especially given the severity of effects of over- and underimmunosuppression. With the current standard-of-care, clinicians rely heavily on intuition and experience to successfully match immunosuppression with rapidly changing patient state and needs. However, PPM has demonstrated ability to retrospectively distinguish between clinically stable patients, infection events, and rejection episode to proactively adjust immunosuppression accordingly. Hence, this novel dosing strategy can alleviate challenges facing standard dosing practices by removing clinician guess work.

This study was conducted retrospectively using prospectively collected data. Future investigations aim to use PPM prospectively in the outpatient setting and evaluate its efficacy for modulating long-term immunosuppression regimes in real time in a practical setting. This will allow for direct comparison against physician-directed, standard-of-care dosing. While PRS functions mathematically bound to ensure returned dosing combinations are appropriate, such a strategy promises to save physicians both time and effort. Given clinically validated outputs that can be optimized, PPM can also be used to optimize immunosuppression for other organs. Furthermore, because PPM functions independently from disease mechanism and drug identity, it is likely to have broader applicability beyond just management of transplant-related immunosuppression regimens. This system can allow clinicians to tailor prescription practices in other combination therapy reliant disciplines such as cancer therapy.

### 5. Examples

This invention is not limited to the particular processes, compositions, or methodologies described, as these may vary. The terminology used in the description is for the purpose of describing the particular versions or embodiments only, and is not intended to limit the scope of the present invention which will be limited only by the appended claims. Although any methods and materials similar or equivalent to those described herein can be used in the practice or testing of embodiments of the present invention, the preferred methods, devices, and materials are now described. All publications mentioned herein, are incorporated by reference in their entirety; nothing herein is to be construed as an admission that the invention is not entitled to antedate such disclosure by virtue of prior invention.

### Example 1. Methods and Procedures

### A. Dd-cfDNA Process

SNP-based methodology used for DNA amplification and measurement of dd-cfDNA is known in the art. Briefly, at each designated time point, two 10 mL tubes of blood were drawn. Samples were processed with an FDA-approved protocol involving isolation and amplification of cfDNA using a massively multiplexed PCR reaction. Amplicons were sequenced on an Illumina™ next-generation sequencing platform. The data were processed, and each case subjected to data review for quality control. Data are reported here as fraction of dd-cfDNA, expressed as a percentage of total cfDNA.

### B. PPM Dosing Process

In certain embodiments of the invention, the practitioner followed a dosing process/regimen as described here: using the fraction of dd-cfDNA as a quantitative measure of allograft injury to be minimized, the PPM process involves taking into consideration the previous dd-cfDNA data, along with the medication regimen history and the clinical scenario such as lab values and presence of infection, to construct a Parabolic Response Surface. The PRS is patient-specific and based on individualized constants that represent each individual patient's response to drug treatment. R-squared analysis was used to calculate the accuracy of the multidimensional second-order polynomial maps. Normal distribution was determined by Shapiro-Wilk normality test. Based on this analysis, the practitioner makes a recommendation to result in a lower fraction of dd-cfDNA. The clinician then adjusts the immunosuppression regimen based on the PPM recommendation and clinical practice guidelines according to the invention.

### C. Data Management and Statistics

The data management system used here was REDCap™. In this protocol, initial examination of data in a patient for a clinical trial includes descriptive statistics, frequency distributions, and histograms in order to identify outliers and missing data and to check data source adequacy. Data is periodically converted to MATLAB, SAS and R data types. Quarterly statistical summaries and progress reports were generated by the statisticians for review by all investigators.

In a clinical study of immunosuppression in kidney transplant, the primary efficacy endpoint is the renal allograft interstitial fibrosis (IF) measured as a continuous variable ranging from 0 to 100%. Preliminary parameters for the sample size justification were obtained from the previous study for treatment groups. Considering an interim analysis, the study size was determined based on group-sequential tests comparing the means of two groups with known standard deviations (STDs). Two stages (one interim and the final stages) and two-sided efficacy with futility (symmetric) were used. The underlying test is the common, known-variance two-sample z-test. Final stage sample size of 85 per group achieves 90% power to detect a mean absolute difference of 6% (relative difference of 28.6%) at a target alpha-level of 0.05. The assumed population means are 21% and 15%, for the standard of care (SOC) and PPM groups at 24 months, respectively, with known STD 12% for both groups. Considering 20% drop-out rate, a total of 212 eligible patients (106 per group) are recruited. The sample size calculation was conducted using the PASS 2020™ software.

### D. Analysis for Primary/Secondary Endpoints

For more advanced analyses, it is useful according to embodiments of the invention to summarize the study patients in a renal transplant treatment trial using descriptive statistics for the demographic and clinical characteristics at baseline by treatment groups. To do this, the mean (STD), median (range) for continuous variables, and frequency (%) for discrete variables were calculated. The primary endpoint (the change in IF measured as a continuous variable at 3 months and 24 months) was explored to check the underlying distributional assumptions by each treatment condition. As needed, log-transformation was considered. In primary analyses, an intent-to-treat approach was used, in which patients were analyzed in their randomized conditions regardless of the number of treatment or number of follow-up assessments they complete. Group comparisons were carried out using two-sided two-sample independent t-test or the corresponding non-parametric approach at each time point.

The general linear regression model for the IFs was fitted with the main covariates of group (SOC vs. PPM), time (3 months & 24 months), and their interaction term. Potential confounding factors, including age, sex, and first versus second transplant, were adjusted for in the model. Furthermore, the potential variability among the sample collection sites was accounted for in the model by including it as a random effect. While both treatment conditions only included patients who have had no rejection on the 3 month biopsy, a sensitivity analysis was conducted to compare the patients who were not included due to rejection with the study patients without evidence of rejection at 3 months. The secondary endpoints are listed in Table 1, below, and each endpoint was analyzed using appropriate statistical tests, including survival analysis for time-to-event data. Effect size and its 95% confidence interval for each endpoint was reported regardless of the statistical significance. All statistical tests were analyzed with a two-sided test in a reproducible manner using SAS 9.4 and R.3.5 software.

### E. Interim Analysis Plan

A group sequential trial with sample sizes of 170 (85 per group) at the final look achieves a 90% power to detect difference in the mean value of IF between the PPT group and SOC group at the 0.05 significance level (Alpha) using a one-sided Z-Test (Pooled). The interim analysis was conducted when a total of 85 patients (43 patients for each group; 50% accumulated information) have completed treatment. The design is based on alpha and beta spending function (O'Brien-Fleming analog), 2000 simulations, binding futility boundaries, 5% target alpha, and two-sided symmetric alternative hypothesis.

### Example 2. Prospective Clinical Trial; Dosing Versus Control

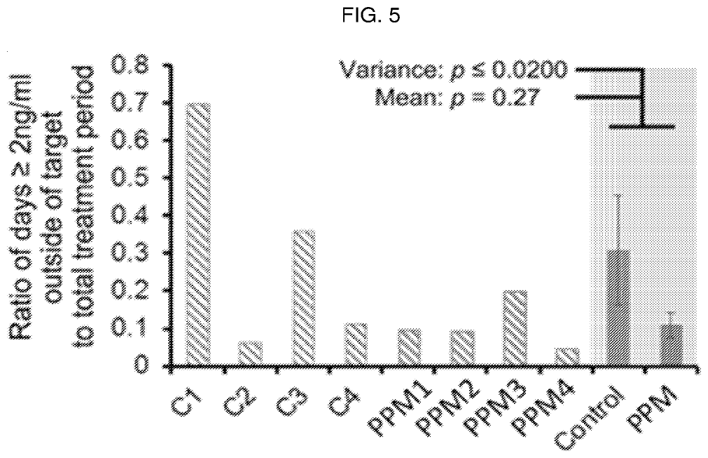

In a first-in-human clinical trial, a prospective clinical pilot study was performed comparing PPM-dosed patients and control (SOC, standard of care dosed) kidney transplant patients. Comparisons between tacrolimus trough levels over the course of treatment showed that PPM dosing markedly improved the management of patient drug levels compared to control patients. See FIG. 5 for results. PPM dosed patients had a lower average ratio of days greater than 2 ng/mL outside of the target range compared control patients.

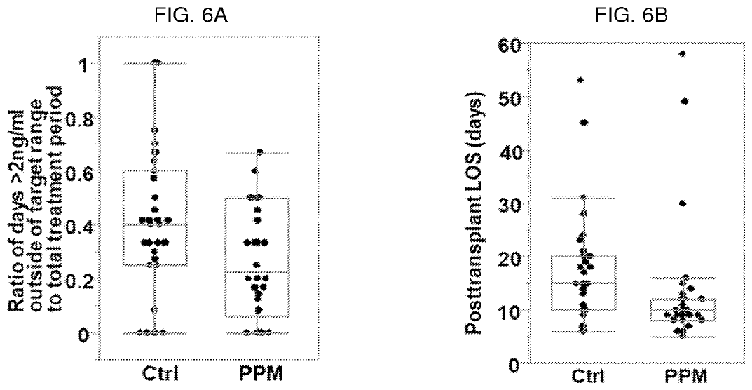

PPM dosing outperformed standard of care dosing in four different comparative tests. These tests included area-under-the-curve analyses, as well as analysis of the frequency and magnitude of deviation from the target range. See FIG. 6. This confirms the conclusions of the previous study. Similar to the pilot study discussed above, subjects with PPM dosing in the larger study (n=61) had (1) a lower fraction of days more than 2 ng/mL outside of the target range (P=0.02) and (2) a shorter length of stay (LOS; P=0.02) compared with standard-of-care dosing. Decreasing the length of stay significantly decreases the costs of the hospitalization.

In summary, the prospective clinical pilot study comparing PPM-dosed patients and control (standard-of-care dosed) patients compared tacrolimus trough levels over the course of treatment and showed that PPM dosing markedly improved the management of patient drug levels compared to control patients. PPM dosing outperformed standard-of-care dosing in four different comparative tests. These tests included area-under-the-curve analyses, as well as analysis of the frequency and magnitude of deviation from the target range (see FIG. 5). A second, larger study confirmed the conclusions of the previous study (see FIG. 6).

This work demonstrated that the methods described here leads to precision tacrolimus administration without the need for mechanistic information by implicitly accounting for genomic, pharmacokinetic, and other patient factors to produce a dosing recommendation. The methods function entirely using phenotypic data (e.g. efficacy/safety or combination of both, which are relatively easy to obtain) to personalize treatment, resulting in a powerful and actionable approach for improved combination therapy.

### Example 3. Multidrug Immunosuppression Dosing in Kidney Transplantation

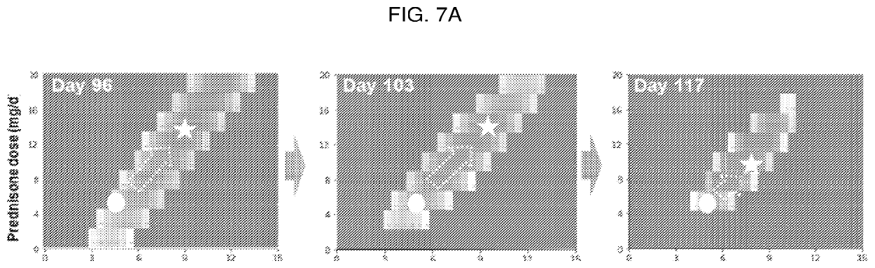

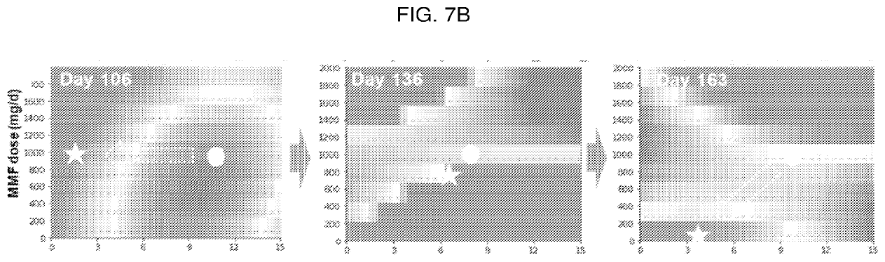

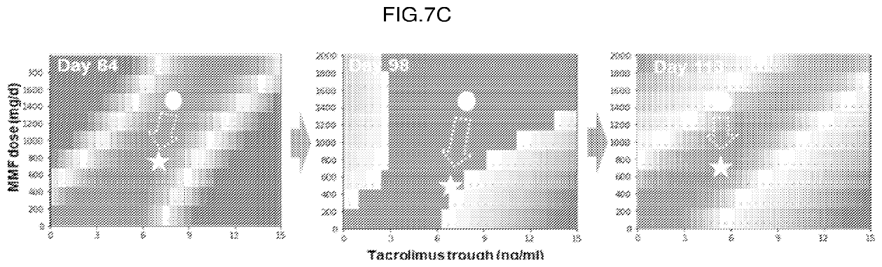

An ongoing retrospective multidrug optimization in kidney transplantation has resulted in reasonable and actionable recommended changes in immunosuppression that can improve the clinical course of patients. See FIG. 7.

FIG. 7A shows that embodiments of the inventive methods consistently recommended increasing immunosuppression in a patient who ultimately suffered from acute rejection. Conversely, embodiments of the inventive methods recommended decreasing immunosuppression in patients who later had recurrent urinary tract infections and ureteral strictures leading to elevated creatinine levels. See FIG. 7B and FIG. 7C, respectively. Circle: actual dose; Star: PPM dose. Blue to red gradient: high to low dd-cfDNA.

### Example 4. Prospective Multicenter Randomized Controlled Artificial Intelligence-Based Immunosuppression Optimization (AIIM) Trial

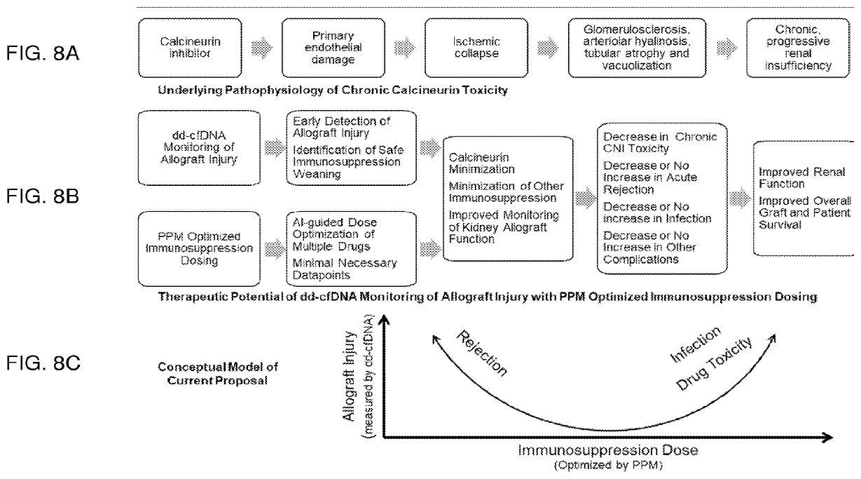

A prospective 23-month multicenter randomized controlled trial evaluates the safety and efficacy of the inventive methods. This particular study is designed without benefit of published data on using an AT or computational approach to the optimization of combination drug therapy in a clinical setting. Based on the evidence summarized in FIG. 8, using dd-cfDNA to monitor transplant injury and to optimize immunosuppression dosing can improve renal function and overall graft and patient survival.

In the study, changes in interstitial fibrosis (IF), glomerular filtration rates, rejection episodes, calcineurin dosing, tubular atrophy and vacuolization, BK viremia, CMV viremia, overall infection rate, HLA and donor-specific antibodies, and graft and patient survival are measured in response to study interventions. Changes in these outcomes are used to understand the effect(s) of the interventions.

The objective of this study is to conserve renal allograft function by optimizing each individual recipient's immunosuppression regimen. Optimization is achieved by minimizing plasma donor-derived cell-free DNA (dd-cfDNA) in kidney recipients, whether caused by over immunosuppression (i.e., toxicity) or under immunosuppression (i.e., rejection). Renal allograft function is assessed by measuring creatinine clearance (CrCl); long-term allograft injury is assessed by quantitating interstitial fibrosis; graft surveillance is achieved by measuring dd-cfDNA. This approach facilitates a safe and effective pathway for prolonging renal allograft function.

Two hundred twelve subjects are recruited at time of transplantation. Inclusion and exclusion criteria are listed below. All subjects are started with institutional standard-of-care: quadruple immunosuppressive therapy with induction (basiliximab or antithymocyte globulin or alemtuzumab), tacrolimus, steroids, and mycophenolate mofetil or mycophenolic acid (MMF or MPA). Maintenance immunosuppression after transplantation also is determined per standard-of-care.

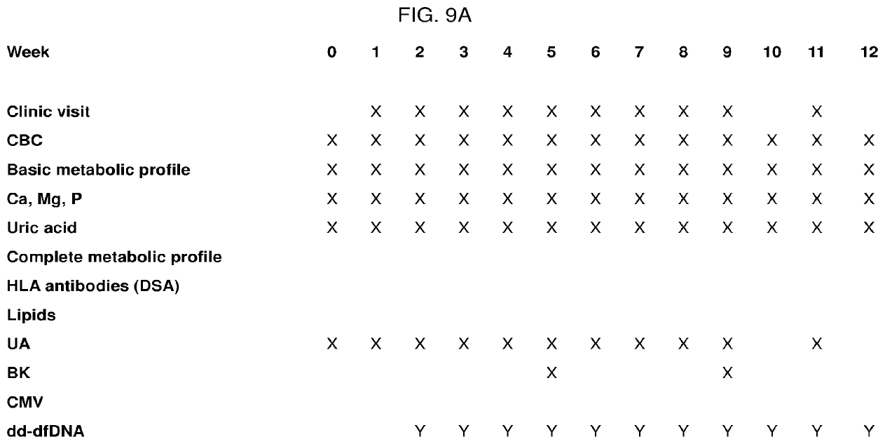

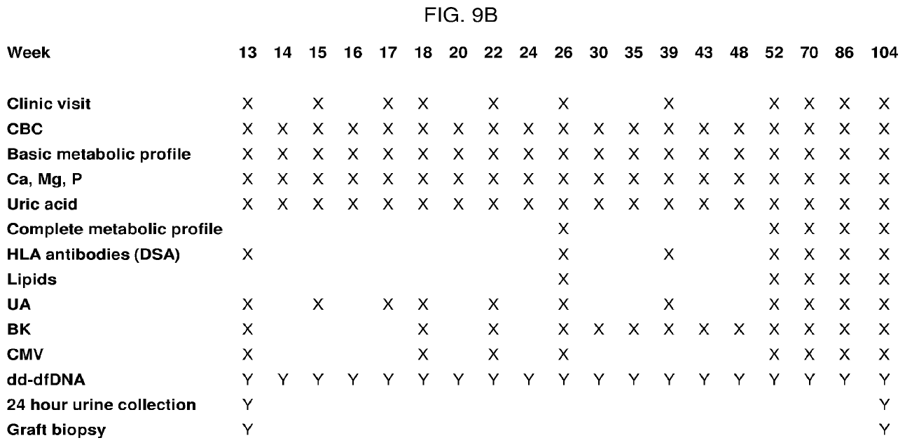

At recruitment, between two weeks and one month after transplantation, subjects are enrolled in the baseline monitoring period of the study. Patients without biopsy proven antibody or T-cell mediated rejection in the first month undergo balanced randomization (1:1) to one of the following treatment arms: (1) standard-of-care per physician preference, or (2) inventive PPM-based dosing. Blood is drawn per a standard-of-care schedule (see FIG. 9). An updated history and physical is obtained, including a full medication history. Biochemical and hematological measurements are recorded at all study visits. Drug exposure of tacrolimus after transplantation is monitored by obtaining trough level measurements as part of standard-of-care labs. dd-cfDNA also is measured according to the schedule below.

After 10 blood draws and no earlier than 3 months after transplantation, a graft biopsy is obtained and analyzed. Patients with evidence of rejection on the 3-month biopsy are excluded as their standard-of-care would not be maintenance immunosuppression and may include a variety of other immunosuppressive medications including thymoglobulin. Patients without rejection (Banff Classification 2018 active or chronic cellular or antibody mediated rejection) continue on to the study.

Control arm subjects continue per SOC, i.e. immunosuppression regimen per center practices. Treatment arm subjects have the dd-cfDNA data analyzed according to an embodiment of the invention. Data, such as drug levels and regimens are used to fit a 2nd order polynomial for each patient to build patient-specific dose-response profiles with covariates that include the administered drugs tacrolimus, steroids, and MMF/MPA. PPM are used to derive an optimal combination of tacrolimus, MMF/MPA, and prednisone to achieve minimal renal allograft injury, while staying within the therapeutic range of the medications. All else being equal, the most efficacious combination with the lowest dose of tacrolimus is used.

If a change is made in the immunosuppression regimen, SOC and dd-cfDNA labs will be obtained one week later to assess for changes and for the regimen to be adjusted accordingly. If no change is made in the immunosuppression regimen, the subject will continue with their SOC labs and clinic visit schedule. A protocol biopsy is performed at the completion of the study at 24 months (21 months after first biopsy).

Table 1 shows the outcome measures planned at baseline and follow-up.

TABLE 1
Data Collection Plan

| | | | 24-month |
|---|---|---|---|
| | | 3-month | (follow- |
| Outcomes | Study Measure | (baseline) | up) |
| Primary | Renal allograft interstitial fibrosis (%) | X | X |
| outcome | | | |
| Secondary | Tubular atrophy and vacuolization | X | X |
| outcomes | Creatinine clearance (24 h urine collection) | X | X |
| | Number & severity of biopsy proven acute rejection | | X |
| | episodes | | |
| | Number & severity of infection episodes | | X |
| | Number & severity of BK viremia episodes | | X |
| | Number & severity of CMV viremia episodes | | X |
| | Activities of de novo HLA class I and II antibodies | X | X |
| | (incl. donor specific antibodies) | | |
| | Number & severity of leukopenia episodes requiring | | X |
| | treatment | | |
| | Number & severity of cardiovascular and/or | | X |
| | metabolic events | | |
| | Cumulative CNI exposure | | X |
| | Daily proteinuria | X | X |
| | Allograft survival | | X |
| | Patient survival | | X |
| Exploratory | Quality of life¹ | X | X |
| outcomes | Medication compliance | X | X |

¹Health related quality of life will be measured with the Short-Form-36 Physical Functioning Score.

### Inclusion Criteria:

1. Patients with end-stage renal disease (ESRD)
2. Adult (18 years of age or older)
3. Recipient of a first or subsequent deceased donor kidney transplant
4. Clinical indication to receive tacrolimus as the primary immunosuppression
5. Subjects willing to provide written informed consent to participate.

### Exclusion Criteria:

1. Recipients of transplanted organs other than kidney
2. Recipients of a transplant from a monozygotic (identical) sibling
3. HLA-identical donor (zero out of six antigen mismatch donor)
4. Recipient of third or more transplant
5. Current or historical panel reactive antibodies of more than 50%
6. Blood Type (ABO) incompatibility or known moderate or strong donor specific antibodies
7. Presence of de novo or recurrent glomerulonephritis on 3-month biopsy
8. Presence of lupus nephritis on 3-month biopsy
9. Presence of focal segmental glomerulosclerosis on 3-month biopsy
10. Presence of BK polyomavirus nephropathy in current or prior transplant
11. Recipients of a bone marrow transplant
12. Recipients who are pregnant
13. Enrollment in a competing trial that would interfere with selection or alteration of immunosuppression
14. Inability to follow up with transplant center for up to 24 months after transplantation
15. Anticipated major surgery during the time of planned study
16. Major medical illness with life expectancy less than 24 months
17. Suspicion of noncompliance
18. Anticipate relocation to a location that would not allow follow up at local center in the next 27 months
19. Inability to tolerate normal range levels of tacrolimus

Previous studies indicate an incidence of graft fibrosis at 6 months after kidney transplant to be approximately 10-12%. After 24 months, the incidence of graft fibrosis in the standard-of-care patients is estimated to be about 20-25%. Thus, a clinically meaningful result correlating with increased creatinine clearance (CrCl) would be about a 25% improvement.

The baseline biopsy at 3 months allows for establishment of an immunological steady state with minimal chronic injury, while at the same time detecting acute injury and diagnosis of any histopathologically apparent graft abnormalities. Subjects with active rejection at 3 months are excluded from the study. Any non-drug related fibrosis can then be detected to allow an accurate comparison between the two groups. The second biopsy allows for more than 20 months of the variable strategies in immunosuppression to have an effect. This is enough time for differences in histopathology to become apparent but likely is not be enough to measure differences in CrCl and graft or recipient survival.

In general, percutaneous kidney biopsies are quite safe with a complication rate below 5%. PPM dosing has been performed in the setting of transplantation in investigational studies by the PI for more than four years. The recommended dose changes are always reviewed. Only recommendations that are within the FDA-approved indications and doses of the medications are used.

Participants who are randomized to the control group are managed per established center protocol. After the immediate postoperative period (14 days), recipients are usually treated with prednisone 5-10 mg daily, mycophenolate mofetil 500-750 mg twice daily, and tacrolimus. The tacrolimus blood trough level goals for standard patients are 8-10 ng/mL until the end of the third postoperative month, 6-8 ng/mL until the end of the sixth month, and 5-7 ng/mL thereafter. Higher risk recipients generally have higher doses per discretion of the prescriber. The clinical team will further incorporate the measurement of dd-cfDNA to inform the management of the immunosuppression regimen per SOC.

Change in renal allograft interstitial fibrosis between 3-month baseline and 24-month follow-up is the primary outcome. Renal allograft interstitial fibrosis (IF) is measured as a continuous variable ranging from 0 to 100%. This outcome 1) correlates well with renal function as measured by CrCl, 2) is a quantitative, continuous, and objective measure thus needing fewer subjects to show a difference between groups, and 3) is less susceptible to acute fluctuations than CrCl and more reflective of chronic injury. All biopsies are evaluated by an expert nephropathologist blinded to the study group.

Another relevant outcome to be measured is CrCl. This can be performed using a 24-hour urine collection method for greatest accuracy. Other outcomes being measured can also be used as markers of allograft function and chronic allograft injury. These include tubular atrophy and vacuolization (as another measure of chronic kidney injury, especially because of CNI toxicity), as well as measurement of daily proteinuria. A powerful early indicator of whether and how the inventive method quantitatively changes the immunosuppression regimen is whether it leads to a change in cumulative tacrolimus exposure. This can be measured both by a summation of total tacrolimus dose administered over the same time period (21 months of study dosing) and by the integration of the cumulative tacrolimus trough levels over the study period.

Less common events such as number and severity of biopsy-proven acute rejection episodes and number and severity of infection episodes are measured, as are episodes of BK virus or cytomegalovirus (CMV) viremia, leukopenia requiring treatment, generation and activities of de novo HLA class I and II donor-specific antibodies. Safety measures such as cardiovascular or metabolic events, though unlikely given the timeframe of the study, also can be examined.

Because safely minimizing immunosuppression is likely also to improve recipient quality of life and adherence to immunosuppression medication, health related quality of life (SF-36 Physical Functioning Score) and medication adherence (Eight-Item Morisky Medication Adherence Scale (MMAS-8)) optionally can be measured.

Participants and their physicians are asked not to change their medications during the study if possible unless otherwise instructed by the investigators. Medications, such as proton pump inhibitors, antihypertensive medicines, and statins, are per SOC. Participants are asked to bring all of their medications to baseline and follow-up visits for recordation of the drug name and dose. Biopsy-proven rejection is treated per SOC (e.g. methylprednisolone pulse and antithymocyte globulin for refractory rejection episodes).

Missing data may occur when some participants are lost to follow-up. First, every effort is made to ensure that participants return for their 24-month follow-up. Proxies can be used to help locate participants that are unable to be reached, and letters can be mailed to participants who do not respond to telephone contacts. Transportation and expenses can be reimbursed to encourage patient compliance. The reason for dropout for each participant; if dropout is completely at random, then the analyses based on available data provide valid statistical inferences. When dropout is not completely at random, several sensitivity analyses can be performed, as much as is possible given the smaller sample size. The multiple imputation approach can be used to account for missing data at the 24-month follow-up under the assumption of missing at random. Additional sensitivity analyses can be used to guard against the possibility of missing data not at random using extreme value imputation, selection models and shared-parameter models.

In summary, the overall goal of the trial is to test the ability of inventive methods to improve kidney function, decrease the need for immunosuppression, decrease episodes of infection and rejection, and improve overall patient quality of life.

### Example 5. Immunosuppression Using AST-Guided Phenotypic Personalized Medicine

Population based dosing strategies fail to account for significant inter- and intra-patient variability, frequently resulting in over- or underimmunosuppression. Variability stems from differences in metabolic phenotypes, epigenetics, and environmental factors, among other reasons. Immunosuppressive drugs have narrow therapeutic ranges and significant risks and side-effects. There is a need for a dosing approach that integrates patient variability. The aim of this study is to use PPM to systematize multidrug immunosuppression regimens in liver transplant recipients. The inputs include the multidrug immunosuppression regimens and responses to clinical outcomes such as AST. Using these inputs, the PRS equation provides doses at desired clinical outcome.

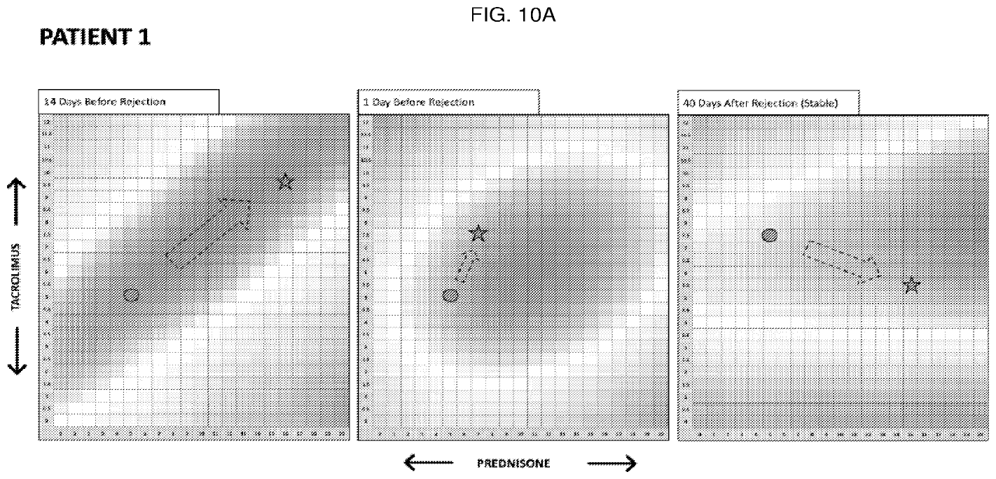

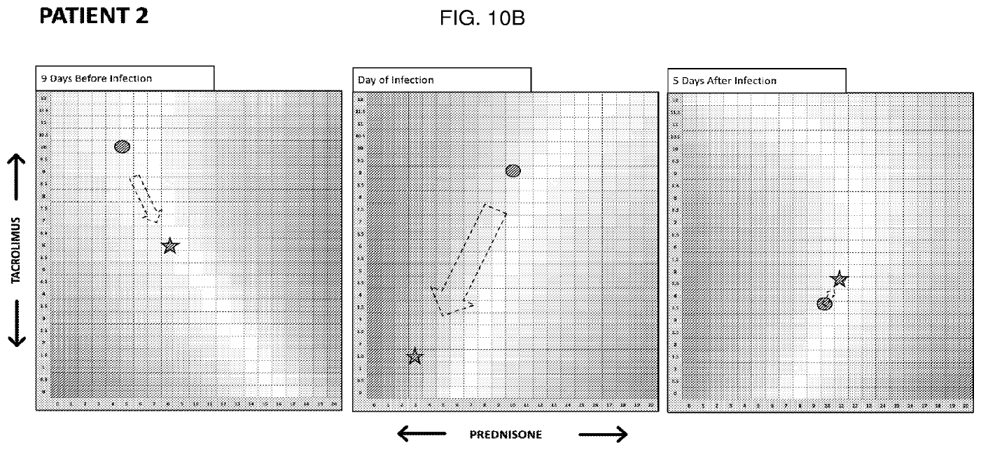

FIG. 10 illustrates the patient timeline and recommendation schedule for 2 separate liver transplant patients: 1 rejection and 1 infection. In both patients, data was used for 2-drug optimization of tacrolimus and prednisone combination dosing. Physician prescribed IS is indicated by the red circle while the PPM recommended dose is indicated by the star. Direction of recommendation is highlighted by the dashed arrow between these two points. The blue-red shading beneath the recommendation vector is a heat map depicting the value of the optimized outcome. In this case, PPM was guided by AST. Hence, areas in red are representative of high AST while areas in blue represent lower AST.

For the rejection patient (FIG. 10, patient 1), PPM was able to recognize need for increased IS 2 weeks before to biopsy confirmation of the episode. Fourteen days before biopsy-proven rejection was diagnosed, PPM recommended increasing tacrolimus from 5.0 mg to 9.5 mg and increasing prednisone from 5.0 mg to 16.0 mg. One day before rejection, PPM recommended increasing tacrolimus from 5.0 mg to 7.5 mg and increasing prednisone from 5.0 mg to 7.0 mg. However, 40 days after the rejection event, when the patient had returned to stable status, PPM recommended decreasing tac from 7.5 mg to 5.5 mg while increasing prednisone from 5.0 mg to 15.0 mg.

For the infection patient (FIG. 10B, patient 2), PPM recognized need for decreased IS 9 days prior to confirmation of infection by positive culture. Nine days before clinical diagnosis of infection, PPM recommended modulation of tacrolimus from 10.0 mg to 6.0 mg and prednisone from 4.5 mg to 8.0 mg, producing an overall decrease in IS. On the day of the infection diagnosis, PPM recommended stronger IS reduction, advising decrease in tacrolimus from 9.0 mg to 1.5 mg and decrease in prednisone from 10.0 to 3.0 mg. During recovery from the event, 5 days post diagnosis, PPM recommended a conservative increase in IS, advising tacrolimus modulation from 4.0 mg to 3.5 mg and prednisone modulation from 10.0 mg to 11.0 mg.

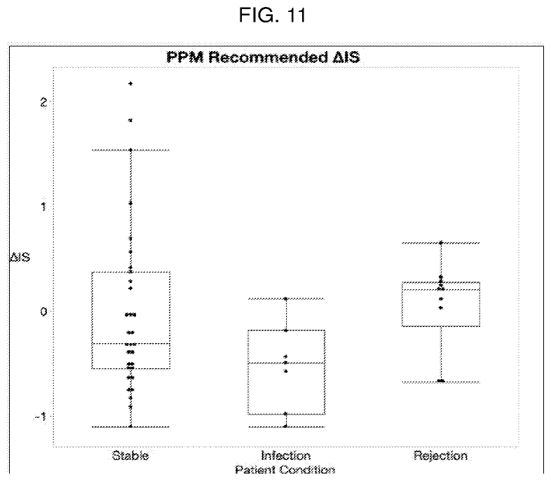

FIG. 11 presents the results of retrospective analysis of 15 liver transplant patients, generating a total of 48 PPM dosing recommendations. See also Table 2, below. On average, analysis of a single patient yielded 3 recommendations. Of such recommendations, 31 were for stable status patients, 7 for infection, and 10 for rejection. In general, PPM demonstrated agreement with clinically advised IS modulation, recommending decreased IS for infection events and increased IS for rejection events. In this case, PPM recommended decreased IS for both stable and infection patients. Average recommended decrease in IS was stronger for infection patients relative to stable, though not significant. Conversely, rejection patients were recommended increased IS. Difference in value of recommended IS for rejection patients is significantly different from stable patients (p=0.0458) and nearly significant relative to infection patients (p=0.0625).

TABLE 2
PPM Recommended ΔIS

| | Mean | Medium | SD |
|---|---|---|---|
| Stable | −0.0288 | −0.3053 | 0.7984 |
| Infection | −0.5210 | −0.4197 | 0.4239 |
| Rejection | 0.0708 | 0.2100 | 0.4207 |

### Example 6. Immunosuppression Optimization Using Dd-cfDNA-Guided Phenotypic Personalized Medicine

### A. Methods

### Patients

Patients were recruited under an IRB approved protocol (IRB202000957) at the University of Florida Health Shands Hospital. In this prospective study, kidney transplant recipients who were more than one month status post kidney transplantation were followed by serial donor-derived cell-free DNA (Prospera or AlloSure) and gene expression analysis (AlloMap). Study blood draws were obtained at regularly scheduled clinic visits at the time of standard-of-care draws, starting at least 30 days after kidney transplantation (to avoid the short-term effects of the transplant operation and induction therapy) and continued up to one year after transplantation. Adjustments to immunosuppression doses were according to standard-of-care and did not take into account results of these studies. Graft function, drug doses, side-effect profiles, and other clinical measures (e.g., donor specific antibodies) were monitored per standard-of-care to correlate with immunosuppression regimen.

### Data Collection

Generation of each dosing recommendation began with collection of two classes of clinical data: treatment inputs (i.e., immunosuppression drug treatment) and treatment output (i.e., measure of allograft injury (dd-cfDNA or transcriptional analysis)). More specifically, each patient's immunosuppression, including tacrolimus blood trough level, and doses of mycophenolate, prednisone, and methylprednisolone were used as treatment inputs. The drug doses/levels were paired with the observed response output, e.g., the fraction of dd-cfDNA measured in the recipient's plasma corresponding to the medication regimen or the transcriptional analysis score.

This allowed for more reproducible accounting for differences in patient drug metabolism. The drug doses/levels were paired with the observed response output, i.e., the fraction of dd-cfDNA measured in the recipient's plasma corresponding to the medication regimen. Inputs were collected beginning 30 days after kidney transplantation and span to between 80 and 300 postoperative days.

### Data Standardization

To standardize the concept of immunosuppression dose, each recorded prescription was first transformed to represent a fraction of a predesignated maximum. Maintaining a consistent dosing scale prevents the introduction of unnecessary error of scale by differences in the order of magnitude of dosing units across medications. Beyond consideration for the viability of maximum and minimum doses, feasibility of recommendations was achieved by confining PPM recommendations to discrete, clinically relevant dosing steps. While inputs may be continuous, falling between defined dosing increments, discrete steps ensure the resulting recommendations are practicable.

Tacrolimus trough level was normalized to a scale from 0-12 ng/mL, with steps of 0.5 ng/mL, resulting in 25 possible trough levels. Hence each 0.5 ng/mL of tacrolimus trough level was 0.0416 normalized units. MMF dose was standardized from 0-2500 mg/day with dosing steps of 250 mg/day, resulting in 11 possible doses. A dosing step of 250 mg/day of MMF was 0.1 normalized units. Finally, prednisone was scaled from 0-20 mg/day, with dosing steps of 0.5 mg/day, resulting in 41 possible doses. Each prednisone dosing step of 0.5 mg/day was 0.025 normalized units. Methylprednisolone dose was converted to prednisone using a 4:5 ratio.

### PPM Dose Optimization

Once transformed, clinical data were processed. PPM integrates each patient's response by relating treatment (immunosuppression doses) with the outcome (dd-cfDNA fraction or transcription score) using a second-order polynomial. In this step, PPM uses this function to approximate the equation that best describes the functional relationship between data points. In other words, PPM adjusts coefficients of the standard equation to achieve a surface that most closely matches the graphical contour of clinical observations. As with other forms of regression, PPM identifies a best fit using sum of squares minimization. The returned function is termed the phenotypic response surface (PRS). The PRS is a mathematical representation of the patient's response to varying levels and combinations of immunosuppressive agents. It demonstrates the association between drug doses and allograft status as measured by the fraction of dd-cDNA in circulation or the transcriptional readout in this study.

Hence, in order to optimize immunosuppression, the PRS was used to identify dosing combinations associated with minimal levels of dd-cfDNA. This was set at 0.12% rather than 0, as this was the lower limit of detection of dd-cfDNA in plasma. For PPM guided by transcription scores, phenotypic inputs ranged in score from 0-40 with lower scores representing a lower likelihood of rejection. Using this method, the PRS was used to identify dosing combinations associated with a transcriptional score of 0. Additionally, solutions are bounded (as described above) to ensure that returned doses are feasible, realistic, and within FDA recommendations.

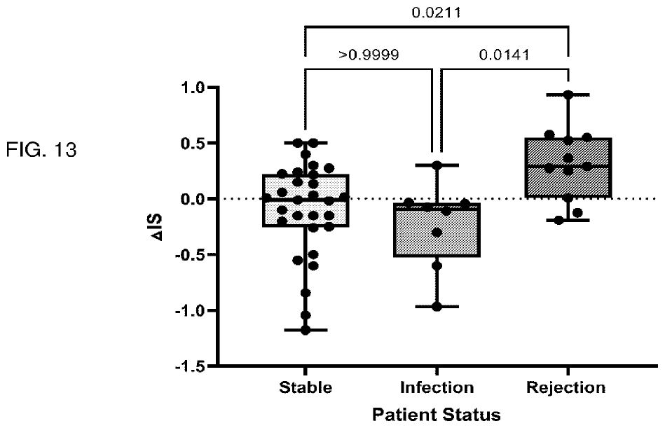

One-drug optimizations (and dosing recommendation) require 3 pairs of treatments and responses; 2-drug optimizations require 6; 3-drug optimizations require 10. Moreover, each drug being optimized should change in dosage at least one time within the set of input data in order to allow the calculation of an optimal dose. A PRS function and corresponding recommendations are then created from the respective number of clinical data points directly preceding the recommendation date. PPM performs a rolling analysis of clinical inputs. Hence, PRS functions are specific not only to each patient but also to the date of recommendation. Use of rolling data allows PPM to accommodate dynamic patient response patterns and changes in immunologic, environmental, physiological, and other variable factors over time (FIG. 13).

The clinical scenario for each dosing recommendation falls into one of three classes: stable, infection or rejection. Assignment to a class depends on the date of the recommendation, which is equivalent to the date of the last treatment and response pair input used to generate the PRS equation. The clinical scenario assignment for the dosing recommendation is based on any events occurring from the day immediately prior to the recommendation and up to 14 days after the recommendation. Stable recommendations are defined by the absence of either rejection or infection events during this two-week window. In this cohort, there were no instances of infection and rejection episodes concurrent within the same window. This assignment strategy tests the ability to analyze predictive power of event related dosing recommendations. PPM analysis progresses from 1-drug to 2-drug to 3-drug optimization, mirroring the increase in the number of required inputs specific to each approach. Optimization of PRS equations constructed from transcriptional phenotypes was performed in the same manner.

Optimization is conducted in two iterations distinguished by the stringency of input data processing. In the first iteration, the quantity of recommendations is maximized at the expense of the quality by assuming constancy of non-input immunosuppressants. This enables use of all available clinical data points. In the second, more stringent iteration, quality of recommendations is favored. In this case, changes in non-input immunosuppressants are not tolerated. Recommendations should be made where only the drugs to be optimized are varying over time.

In either case, PPM analysis progresses from 1-drug to 2-drug to 3-drug optimization, mirroring the increase in the number of required inputs specific to each approach. Because 1-drug optimization requires the fewest pairs of treatment inputs, it is applied to the earlier portions of the patient timeline. Conversely, at later time points, with greater numbers of accumulated inputs, 2-drug and 3-drug optimizations are favored respectively. Overall, analysis is dominated by 2-drug optimization as it is less data-intensive than 3-drug optimization but better able to account for synergistic effects between drugs than 1-drug optimization.

In all cases, models were fit appropriately so that the PRS equation was able to identify dosing combinations where the projected dd-cfDNA fraction was closest to the aimed value of 0.12%. This demonstrated the efficacy of the function's basic mechanism to seek the ideal phenotype defined within a protocol.

To further validate the model, the errors in the predictions made by PRS functions were measured. The error represents the difference between the dd-cfDNA fraction predicted by PRS for the actual prescribed drug combination and the dd-cfDNA fraction observed clinically given that actual combination. The mean error for all recommendations was 2.86%. No significant differences in PRS function error were observed between stable, infection, and rejection groups.

### Strength of PPM Recommendation

The strength of the recommendation was calculated by taking the length of the PPM vector, which describes the difference between the patient's prescribed dose combination and the dose combination suggested by the PRS function. Since the PRS functions are generated using the aforementioned standardized scale, changes in IS are determined independently of differences in prescription unit and are again quantified relative to the maximum daily dose. However, the direction of change along each immunosuppressant axis is binary and often, PPM generated dosing combinations indicate simultaneous upward and downward modulation. To account for the multi-dimensional nature of recommendations, strength is represented by the magnitude of the net vector, where each component denotes the change in a single immunosuppressant necessary to achieve PPM's target dosing values. While the sum or average of components is susceptible to overstate the overall change given modulation along multiple axes, magnitude indicates the displacement between the prescribed dosing combination and the dosing combination recommended by the PRS function.

### Direction of PPM Recommendation

Direction describes the overall change in immunosuppression, whether increasing or decreasing relative to the prescribed IS regime. Direction was determined by the sign, positive or negative, of the total recommendation vector. There were no instances in which PPM identified dosing combinations were identical to the patient's prescribed regimen or the overall value of immunosuppression was equal to that prescribed by the clinician. Hence, in the analysis here, direction was binary.

### B. Results

### Patient Population & Characteristics

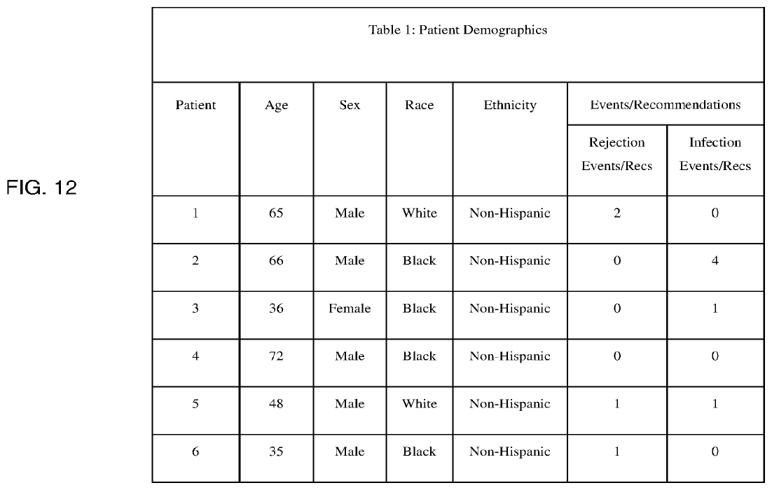

Between Oct. 14, 2020, and Feb. 20, 2020, 32 patients were recruited. Retrospective PPM analysis was performed on data from 6 kidney transplant recipients. The remaining patients did not have enough dd-cfDNA or transcriptional data to yield informative recommendations. For these participants, either the number of collections was insufficient to meet PPM requirements, or the dates of collection were too far apart (any concrete number here?) to produce meaningful data. See FIG. 12.

Differences in magnitude of rejection recommendation vectors were significantly from both stable (p=0.021) and infection (p=0.014) groups. Hence, PRS functions were able to reflect changes occurring in patient phenotypes prior to rejection events and recognize need for upward modulation up to two weeks before the diagnosis of rejection. However, the magnitudes of stable and infection recommendation vectors were not significantly different (p>0.99), despite advising stronger immunosuppression reduction to infection patients relative compared to stable patients.

### C. Donor-Derived Cell-Free DNA

### Quantity Favored Approach

It was possible to make a total of 48 dosing recommendations. Of these, 29 were classified as recommendations for stable future timepoints, 8 as future infections, and 11 as future rejections. The direction of PPM suggested immunosuppression modulation aligned with standard clinical care practices in 16 of the 19 adverse event-related recommendations (9 of 11 rejection recommendations and 7 of 8 infections recommendations). In these instances, infection patients were recommended decreased IS, while rejection patients were recommended increased immunosuppression. Stable status recommendations were split in direction with 15 of 19 recommending decreased IS and 14 of 19 recommended increased IS. More specifically, for patients experiencing infection events, PPM recommended IS change of −0.23 (SD 0.39) normalized units, a reduction. For patients experiencing rejection events, PPM advised IS change of +0.31 (SD 0.33) normalized units, an increase. Finally, for recommendations made for patients in stable status windows, PPM generally recommended a change in IS −0.10 (SD 0.43) normalized units. See FIG. 13, a boxplot of PPM recommended changes in immunosuppression according to recommendation class.

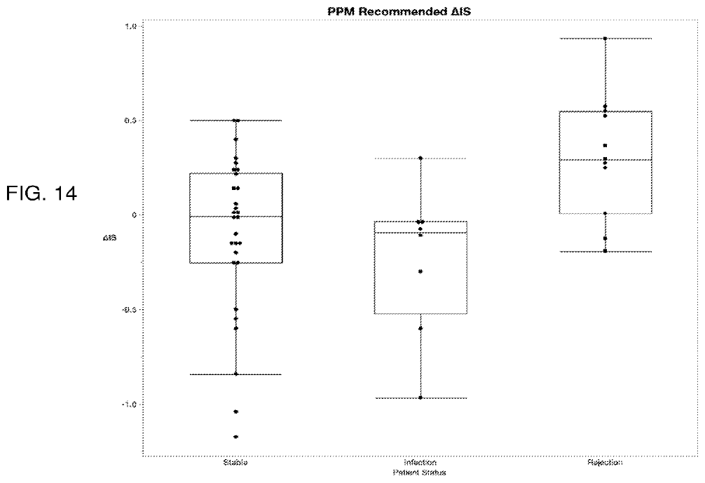

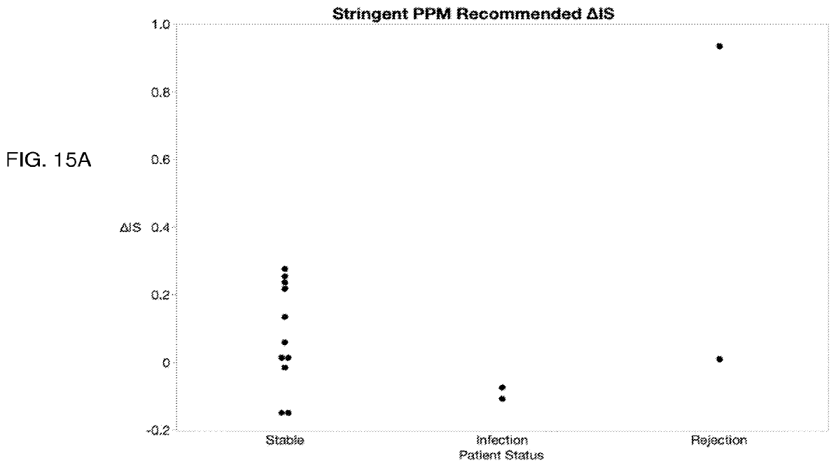

The direction of PPM suggested immunosuppression modulation aligned with standard clinical care practices for both event related groups, advising decreased immunosuppression for infection events and increased immunosuppression for rejection events. More specifically, PPM recommended IS reduction of mean 0.2281 and median 0.0917 normalized units during infection events. For rejection recommendations, PPM advised IS increases with a mean of 0.3144 and median of 0.2917 normalized units. Finally, for recommendations made in stable patient status windows, PPM generally recommended a reduction in IS with mean of −0.1011 and median of 0.0083 normalized units. The results are visualized in FIG. 14.

The direction of PPM suggested immunosuppression modulation aligned with standard clinical care practices in 16 of the 19 adverse event-related recommendations (9 of 11 rejection recommendations and 7 of 8 infections recommendations). In these instances, infection patients were recommended decreased IS while rejection patients were recommended increased immunosuppression. Stable status recommendations were split in direction with 15 of 19 recommending decreased IS and 14 of 19 recommended increased IS.

The strength of the recommendations was significantly different among stable, infection, and rejection groups. Though PPM recommended increased immunosuppression during both stable and rejection statuses, PRS functions found need for more aggressive upward modulation in rejection patients. Based on interquartile ranges, PPM recommended an increase between 7.5% and 17.5% for stable patients and an increase between 42.1% and 72.1% for rejection patients. For the infection group PPM recommended between an immunosuppression decrease between 3.8% and 28.2%. Differences in strength of recommendation were most highly significant between stable and rejection groups though all pairwise comparisons showed significant differences.

### Quality Favored Approach

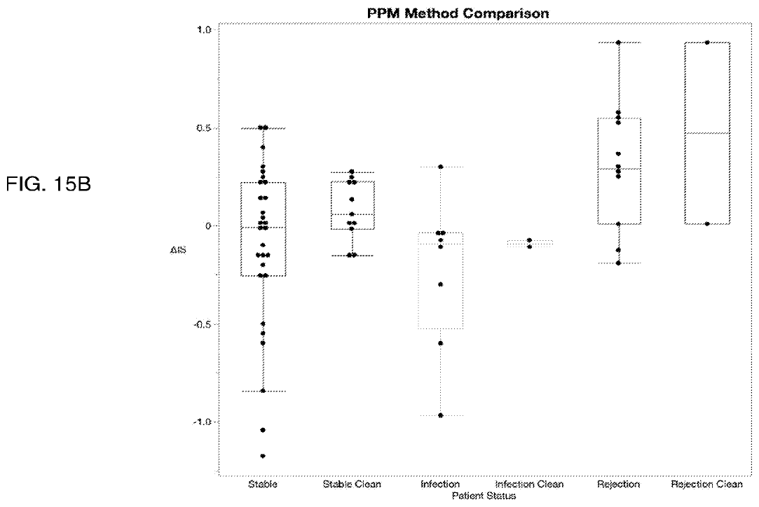

After conducting initial complete coverage analysis, stringent filtration criteria were applied to as described to score quality favored recommendations. This approach yielded 15 total dosing recommendations; 11 were classified as stable, 2 as infection and 2 as rejection related. For stable patients PPM recommended increased IS with mean 0.0780 and median 0.0583 normalized units (FIG. 15).

### Gene Expression Analysis

PPM is adaptable to a variety of clinical inputs. In addition, cell-free DNA measurements (AlloSure), the ability of gene expression analysis (AlloMap) was evaluated for guiding the model for IS management. Aside from input identity, methods were otherwise consistent in regard to number of inputs, identification of adverse event windows, and PRS equation formulations.

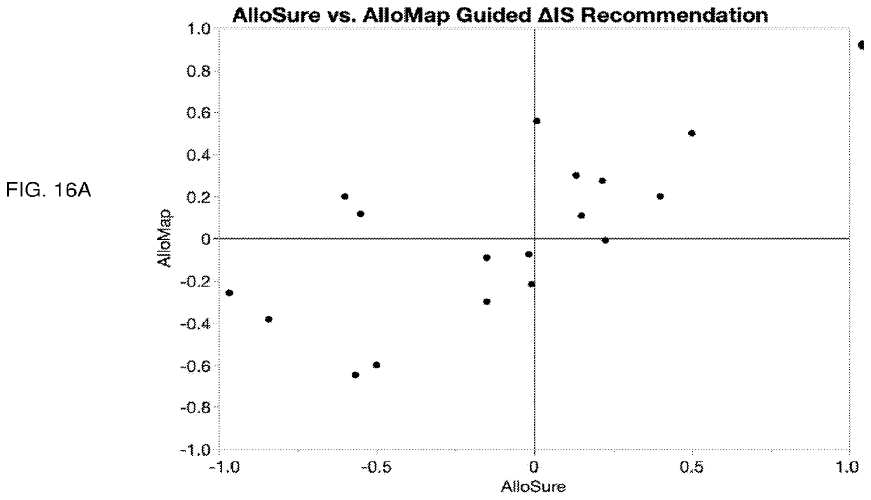

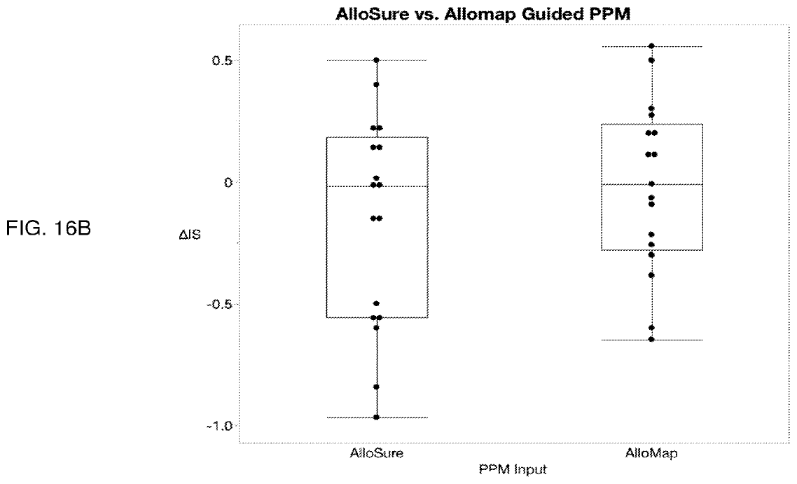

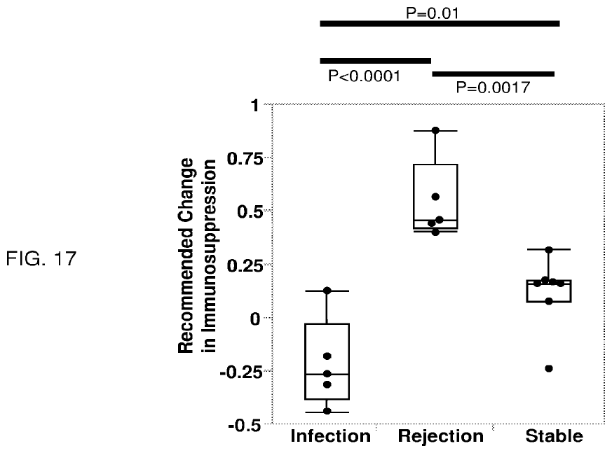

Using a subset of 3 patients from the full study's cohort, PPM was applied to generate 17 total dosing recommendations, 1 of which was predictive of infection. No further infection or rejection recommendations could be made using the AlloMap patient subset. AlloSure and AlloMap recommendations were compared in both strength and direction. FIG. 16 shows each comparable recommendation date with calculated change in immunosuppression guided by dd-cfDNA (Allosure) and gene expression analysis (AlloMap) respectively. Of the 17 recommendations 14 were in directional agreement with AlloSure generated recommendations. All 3 recommendations conflicting between input methods were for stable patients. There were no significant differences in the value of IS change recommended (p=0.4908) between AlloMap and AlloSure PPM methods (FIG. 16, FIG. 17).

### Validation of PPM Recommendations

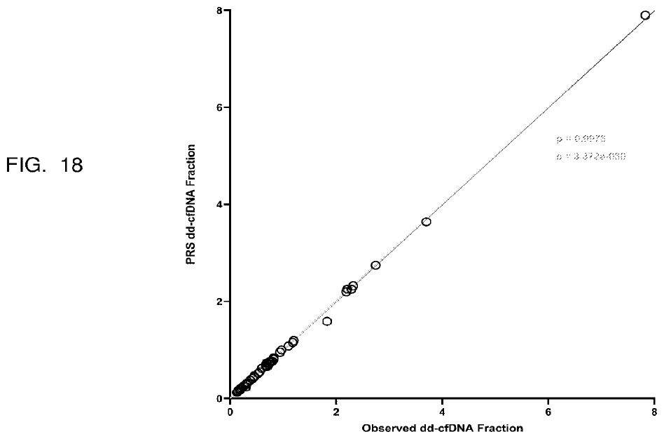

The mean error for all recommendations was 2.64%. The median error was 1.19%. No significant differences in PRS function error were observed between stable, infection, and rejection groups. The PRS modeled phenotype was statistically correlated to the clinically observed phenotype with coefficient ρ=0.9973. See FIG. 18, a scatter plot of patients measured dd-cfDNA fraction versus the dd-cfDNA fraction projected by PRS functions at the physician prescribed dosing combination. Error is represented by the difference between the observed and modeled phenotypes.

### Gene Expression Based Recommendations

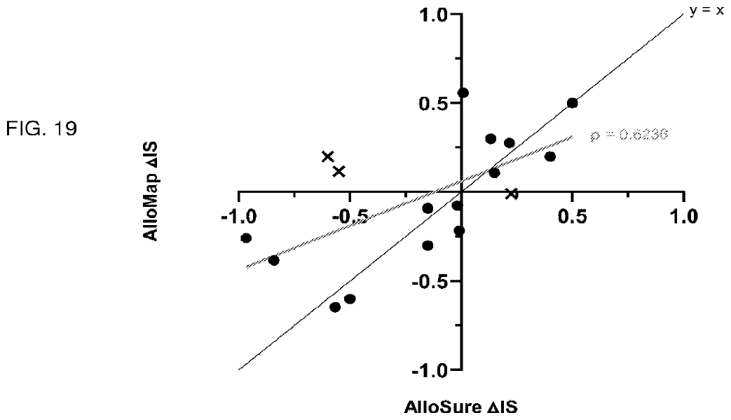

PPM is adaptable to a variety of clinical inputs. In addition to donor-derived cell-free DNA measurements, the ability of gene expression analysis (AlloMap) to guide the model for IS management was evaluated. Aside from the output measure, methods were otherwise consistent in regard to number of inputs, identification of adverse event windows, and PRS equation formulations. AlloMap data were available for 3 patients from the full study cohort. PPM were applied to generate 17 total dosing recommendations, 1 of which predicted infection. No further infection or rejection recommendations could be made using the AlloMap patient subset. See FIG. 19, which shows the recommended change in immunosuppression guided by dd-cfDNA fraction (AlloSure) vs. gene expression analysis score (AlloMap).

AlloSure and AlloMap recommendations were compared in both strength and direction. FIG. 19 shows each comparable recommendation date with calculated change in immunosuppression guided by dd-cfDNA (AlloSure) and gene expression analysis (AlloMap) respectively. Of the 17 recommendations, 14 were in directional agreement with AlloSure generated recommendations. All 3 recommendations conflicting between input methods were for stable patients. There were no significant differences in the value of IS change recommended (p=0.49) between AlloMap and AlloSure PPM methods. Value of advised IS modulation using AlloMap and AlloSure inputs were correlated with coefficient ρ=0.9973 and p=0039.

### REFERENCES

All references listed below and throughout the specification are hereby incorporated by reference in their entirety.
1. Zarrinpar A, Busuttil R W. Liver transplantation: past, present and future. Nat Rev Gastroenterol Hepatol. 2013; 10(7):434-40. doi: 10.1038/nrgastro.2013.88. PubMed PMID: 23752825.
2. Starzl T E, Iwatsuki S, Shaw B W, Jr., Gordon R D, Esquivel C O. Immunosuppression and other nonsurgical factors in the improved results of liver transplantation. Seminars in liver disease. 1985; 5(4):334-43. doi: 10.1055/s-2008-1040630. PubMed PMID: 3909427; PMCID: PMC2975458.
3. Tanzi M G, Undre N, Keirns J, Fitzsimmons W E, Brown M, First M R. Pharmacokinetics of prolonged-release tacrolimus and implications for use in solid organ transplant recipients. Clinical transplantation. 2016; 30(8):901-11. doi: 10.1111/ctr.12763. PubMed PMID: 27220013.
4. Marcén R. Immunosuppressive drugs in kidney transplantation: impact on patient survival, and incidence of cardiovascular disease, malignancy and infection. Drugs. 2009; 69(16):2227-43. doi: 10.2165/11319260-000000000-00000. PubMed PMID: 19852526.
5. Ojo A O, Held P J, Port F K, Wolfe R A, Leichtman A B, Young E W, Arndorfer J, Christensen L, Merion R M. Chronic renal failure after transplantation of a nonrenal organ. N Engl J Med. 2003; 349(10):931-40. Epub 2003/09/05. doi: 10.1056/NEJMoa021744. PubMed PMID: 12954741.
6. Williams D, Haragsim L. Calcineurin nephrotoxicity. Adv Chronic Kidney Dis. 2006; 13(1):47-55. doi: 10.1053/j.ackd.2005.11.001. PubMed PMID: 16412970.
7. di Paolo S, Teutonico A, Stallone G. Infante B, Schena A, Grandaliano G, Battaglia M, Ditonno P, Schena P F. Cyclosporin exposure correlates with 1 year graft function and histological damage in renal transplanted patients. Nephrol Dial Transplant. 2004; 19(8):2107-12. Epub 2004/06/08. doi: 10.1093/ndt/gfh344. PubMed PMID: 15187199.
8. Fortin M C, Raymond M A, Madore F, Fugère J A, Pâquet M, St-Louis G, Hébert M J. Increased risk of thrombotic microangiopathy in patients receiving a cyclosporin-sirolimus combination. Am J Transplant. 2004; 4(6):946-52. doi: 10.1111/j.1600-6143.2004.00428.x. PubMed PMID: 15147429.
9. A comparison of tacrolimus (FK 506) and cyclosporine for immunosuppression in liver transplantation. The U.S. Multicenter FK506 Liver Study Group. N Engl J Med. 1994; 331(17):1110-5. doi: 10.1056/NEJM199410273311702. PubMed PMID: 7523946.
10. Ekberg H, Grinyó J, Nashan B, Vanrenterghem Y, Vincenti F, Voulgari A, Truman M, Nasmyth-Miller C, Rashford M. Cyclosporine sparing with mycophenolate mofetil, daclizumab and corticosteroids in renal allograft recipients: the CAESAR Study. Am J Transplant. 2007; 7(3):560-70. Epub 2007/01122. doi: 10.1111/j.1600-6143.2006.01645.x. PubMed PMID: 17229079.
11. Ekberg H, Bernasconi C, Tedesco-Silva H, Vítko S, Hugo C, Demirbas A, Acevedo R R, Grinyó J, Frei U, Vanrenterghem Y, Daloze P, Halloran P. Calcineurin inhibitor minimization in the Symphony study: observational results 3 years after transplantation. Am J Transplant. 2009; 9(8):1876-85. Epub 2009/06/26. doi: 10.1111/j.1600-6143.2009.02726.x. PubMed PMID: 19563339.
12. Holdaas H, Rostaing L, Serón D, Cole E, Chapman J, Fellstrøm B, Strom E H, Jardine A, Midtvedt K, Machein U, Ulbricht B, Karpov A, O'Connell P J, Investigators A. Conversion of long-term kidney transplant recipients from calcineurin inhibitor therapy to everolimus: a randomized, multicenter, 24-month study. Transplantation. 2011; 92(4):410-8. doi: 10.1097/TP.0b013e318224c12d. PubMed PMID: 21697773.
13. Naesens M, Kuypers D R, Sarwal M. Calcineurin inhibitor nephrotoxicity. Clin J Am Soc Nephrol. 2009; 4(2):481-508. doi: 10.2215/CJN.04800908. PubMed PMID: 19218475.
14. Sharif A, Shabir S, Chand S, Cockwell P, Ball S, Borrows R. Meta-analysis of calcineurin-inhibitor-sparing regimens in kidney transplantation. J Am Soc Nephrol. 2011; 22(11):2107-18. Epub 2011/09/23. doi: 10.1681/ASN.2010111160. PubMed PMID: 21949096; PMCID: PMC3280000.
15. Ekberg H, Tedesco-Silva H, Demirbas A, Vítko S, Nashan B, Gürkan A, Margreiter R, Hugo C, Grinyó J M, Frei U, Vanrenterghem Y, Daloze P, Halloran P F, Study E-S. Reduced exposure to calcineurin inhibitors in renal transplantation. N Engl J Med. 2007; 357(25):2562-75. doi: 10.1056/NEJMoa067411. PubMed PMID: 18094377.
16. Evenepoel P, Lerut E, Naesens M, Bammens B, Claes K, Kuypers D, Vermeersch P, Meijers B, Van Damme B, Vanrenterghem Y. Localization, etiology and impact of calcium phosphate deposits in renal allografts. Am J Transplant. 2009; 9(11):2470-8. Epub 2009/08/14. doi: 10.1111/j.1600-6143.2009.02792.x. PubMed PMID: 19681815.
17. Issa N, Kukla A, Ibrahim H N. Calcineurin inhibitor nephrotoxicity: a review and perspective of the evidence. Am J Nephrol. 2013; 37(6):602-12. Epub 2013/06/18. doi: 10.1159/000351648. PubMed PMID: 23796509.
18. Schena F P, Pascoe M D, Alberu J, del Carmen Rial M, Oberbauer R, Brennan D C, Campistol J M, Racusen L, Polinsky M S, Goldberg-Alberts R, Li H, Scarola J, Neylan J F, Group SCTS. Conversion from calcineurin inhibitors to sirolimus maintenance therapy in renal allograft recipients: 24-month efficacy and safety results from the CONVERT trial. Transplantation. 2009; 87(2):233-42. doi: 10.1097/TP.0b013e3181927a41. PubMed PMID: 19155978.
19. Budde K, Lehner F, Sommerer C, Reinke P, Arns W, Eisenberger U, Wuthrich R P, Mohlfeld A, Heller K, Porstner M, Veit J, Paulus E M, Witzke O, Investigators ZS. Five-year outcomes in kidney transplant patients converted from cyclosporine to everolimus: the randomized ZEUS study. Am J Transplant. 2015; 15(1):119-28. Epub 2014/12/17. doi: 10.1111/ajt.12952. PubMed PMID: 25521535.
20. Asare A, Kanaparthi S, Lim N, Phippard D, Vincenti F, Friedewald J, Pavlakis M, Poggio E, Heeger P, Mannon R, Burrell B E, Morrison Y, Bridges N, Sanz I, Chandraker A, Newell K A, Turka L A. B Cell Receptor Genes Associated With Tolerance Identify a Cohort of Immunosuppressed Patients With Improved Renal Allograft Graft Function. Am J Transplant. 2017; 17(10):2627-39. Epub 2017/05/02. doi: 10.1111/ajt.14283. PubMed PMID: 28371372; PMCID: PMC5620117.
21. Dugast E, Soulillou J P, Foucher Y, Papuchon E, Guerif P, Paul C, Riochet D, Chesneau M, Cesbron A, Renaudin K, Dantal J, Giral M, Brouard S. Failure of Calcineurin Inhibitor (Tacrolimus) Weaning Randomized Trial in Long-Term Stable Kidney Transplant Recipients. Am J Transplant. 2016; 16(11):3255-61. Epub 2016/07/29. doi: 10.1111/ajt.13946. PubMed PMID: 27367750.
22. Karpe K M, Talaulikar G S, Walters G D. Calcineurin inhibitor withdrawal or tapering for kidney transplant recipients. Cochrane Database Syst Rev. 2017; 7:CD006750. Epub 2017/07/21. doi: 10.1002/14651858.CD006750.pub2. PubMed PMID: 28730648; PMCID: PMC6483545.
23. Meier-Kriesche H U, Schold J D, Srinivas T R, Kaplan B. Lack of improvement in renal allograft survival despite a marked decrease in acute rejection rates over the most recent era. Am J Transplant. 2004; 4(3):378-83. doi: 10.1111/j.1600-6143.2004.00332.x. PubMed PMID: 14961990.
24. Bemelman F J, de Fijter J W, Kers J, Meyer C, Peters-Sengers H, de Maar E F, van der Pant K A, de Vries A P, Sanders J S, Zwinderman A, Idu M M, Berger S, Reinders M E, Krikke C, Bajema I M, van Dijk M C, Ten Berge I J, Ringers J, Lardy J, Roelen D, Moes D J, Florquin S, Homan van der Heide J J. Early Conversion to Prednisolone/Everolimus as an Alternative Weaning Regimen Associates With Beneficial Renal Transplant Histology and Function: The Randomized-Controlled MECANO Trial. Am J Transplant. 2017; 17(4):1020-30. Epub 2016/10/17. doi: 10.1111/ajt.14048. PubMed PMID: 27639190.
25. Cooney G F, Fiel S B, Shaw L M, Cavarocchi N C. Cyclosporine bioavailability in heart-lung transplant candidates with cystic fibrosis. Transplantation. 1990; 49(4):821-3. PubMed PMID: 2326880.
26. Uesugi M, Masuda S, Katsura T, Oike F, Takada Y, Inui K. Effect of intestinal CYP3A5 on postoperative tacrolimus trough levels in living-donor liver transplant recipients. Pharmacogenet Genomics. 2006; 16(2):119-27. PubMed PMID: 16424824.
27. Yu S, Wu L, Jin J, Yan S, Jiang G, Xie H, Zheng S. Influence of CYP3A5 gene polymorphisms of donor rather than recipient to tacrolimus individual dose requirement in liver transplantation. Transplantation. 2006; 81(1):46-51. PubMed PMID: 16421475.
28. Lodewijk L, Mall A, Spearman C W, Kahn D. Effect of liver regeneration on the pharmacokinetics of immunosuppressive drugs. Transplantation proceedings. 2009; 41(1):379-81. doi: 10.1016/j.transproceed.2008.10.055. PubMed PMID: 19249561.
29. Kravljaca M, Perovic V, Pravica V, Brkovic V, Milinkovic M, Lausevic M, Naumovic R. The importance of MDR1 gene polymorphisms for tacrolimus dosage. Eur J Pharm Sci. 2016; 83:109-13. doi: 10.1016/j.ejps.2015.12.020. PubMed PMID: 26705892.
30. Wong P K, Yu F, Shahangian A, Cheng G, Sun R, Ho C M. Closed-loop control of cellular functions using combinatory drugs guided by a stochastic search algorithm. Proceedings of the National Academy of Sciences of the United States of America. 2008; 105(13):5105-10. doi: 10.1073/pnas.0800823105. PubMed PMID: 18356295; PMCID: PMC2278193.
31. Dengu F. Next-generation sequencing methods to detect donor-derived cell-free DNA after transplantation. Transplant Rev (Orlando). 2020:100542. Epub 2020/03/16. doi: 10.1016/j.trre.2020.100542. PubMed PMID: 32265093.
32. Bloom R D, Bromberg J S, Poggio E D, Bunnapradist S, Langone A J, Sood P, Matas A J, Mehta S, Mannon R B, Sharfuddin A, Fischbach B, Narayanan M, Jordan S C, Cohen D, Weir M R, Hiller D, Prasad P, Woodward R N, Grskovic M, Sninsky J J, Yee J P, Brennan D C, Investigators CD-DC-FDiBfDARiKTRDS. Cell-Free DNA and Active Rejection in Kidney Allografts. J Am Soc Nephrol. 2017; 28(7):2221-32. Epub 2017/03/09. doi: 10.1681/ASN.2016091034. PubMed PMID: 28280140; PMCID: PMC5491290.
33. Huang E, Sethi S, Peng A, Najjar R, Mirocha J, Haas M, Vo A, Jordan S C. Early clinical experience using donor-derived cell-free DNA to detect rejection in kidney transplant recipients. Am J Transplant. 2019; 19(6):1663-70. Epub 2019/03/29. doi: 10.1111/ajt.15289. PubMed PMID: 30725531.
34. Al-Shyoukh I, Yu F, Feng J, Yan K, Dubinett S, Ho C M, Shamma J S, Sun R. Systematic quantitative characterization of cellular responses induced by multiple signals. BMC Syst Biol. 2011; 5:88. Epub 2011/05/30. doi: 10.1186/1752-0509-5-88. PubMed PMID: 21624115; PMCID: PMC3138445.
35. Ho D, Wang C H, Chow E K. Nanodiamonds: The intersection of nanotechnology, drug development, and personalized medicine. Sci Adv. 2015; 1(7):e1500439. doi: 10.1126/sciadv.1500439. PubMed PMID: 26601235; PMCID: PMC4643796.
36. Wang H, Lee D K, Chen K Y, Chen J Y, Zhang K, Silva A, Ho C M, Ho D. Mechanism-independent optimization of combinatorial nanodiamond and unmodified drug delivery using a phenotypically driven platform technology. ACS Nano. 2015; 9(3):3332-44. doi: 10.1021/acsnano.5b00638. PubMed PMID: 25689511.
37. Silva A, Lee B Y, Clemens D L, Kee T, Ding X, Ho C M, Horwitz M A. Output-driven feedback system control platform optimizes combinatorial therapy of tuberculosis using a macrophage cell culture model. Proceedings of the National Academy of Sciences of the United States of America. 2016; 113(15):E2172-9. doi: 10.1073/pnas.1600812113. PubMed PMID: 27035987; PMCID: PMC4839402.
38. Nowak-Sliwinska P, Weiss A, Ding X, Dyson P J, van den Bergh H, Griffioen A W, Ho C M. Optimization of drug combinations using Feedback System Control. Nat Protoc. 2016; 11(2):302-15. doi: 10.1038/nprot.2016.017. PubMed PMID: 26766116.
39. Weiss A, Ding X, van Beijnum J R, Wong I, Wong T J, Berndsen R H, Dormond O, Dallinga M, Shen L, Schlingemann R O, Pili R, Ho C M, Dyson P J, van den Bergh H, Griffioen A W, Nowak-Sliwinska P. Rapid optimization of drug combinations for the optimal angiostatic treatment of cancer. Angiogenesis. 2015; 18(3):233-44. doi: 10.1007/s10456-015-9462-9. PubMed PMID: 25824484; PMCID: PMC4473022.
40. Mohd Abdul Rashid M B, Toh T B, Silva A, Nurrul Abdullah L, Ho C M, Ho D, Chow E K. Identification and Optimization of Combinatorial Glucose Metabolism Inhibitors in Hepatocellular Carcinomas. J Lab Autom. 2015; 20(4):423-37. doi: 10.1177/2211068215579612. PubMed PMID: 25824204.
41. Zarrinpar A, Lee D K, Silva A, Datta N, Kee T, Eriksen C, Weigle K, Agopian V, Kaldas F, Farmer D, Wang S E, Busuttil R, Ho C M, Ho D. Individualizing liver transplant immunosuppression using a phenotypic personalized medicine platform. Science translational medicine. 2016; 8(333):333ra49. doi: 10.1126/scitranslmed.aac5954. PubMed PMID: 27053773.
42. Lee D K, Chang V Y, Kee T, Ho C M, Ho D. Optimizing Combination Therapy for Acute Lymphoblastic Leukemia Using a Phenotypic Personalized Medicine Digital Health Platform: Retrospective Optimization Individualizes Patient Regimens to Maximize Efficacy and Safety. SLAS Technol. 2017; 22(3):276-88. Epub 2016/12/13. doi: 10.1177/2211068216681979. PubMed PMID: 27920397.
43. Zarrinpar A, Lee C, Noguchi E, Yersiz H, Agopian V G, Kaldas F M, Farmer D G, Busuttil R W. A rapid, reproducible, noninvasive predictor of liver graft survival. J Surg Res. 2015; 197(1):183-90. Epub 2015/05/06. doi: 10.1016/j.jss.2015.03.093. PubMed PMID: 25940156; PMCID: PMC5025251.
44. Zarrinpar A, Dutson E P, Mobley C, Busuttil R W, Lewis C E, Tillou A, Cheaito A, Hines O J, Agopian V G, Hiyama D T. Intraoperative Laparoscopic Near-Infrared Fluorescence Cholangiography to Facilitate Anatomical Identification: When to Give Indocyanine Green and How Much. Surg Innov. 2016; 23(4):360-5. Epub 2016/03/12. doi: 10.1177/1553350616637671. PubMed PMID: 26964557.
45. Angarita S A K, Duarte S, Russell T A, Ruchala P, Elliott I A, Whitelegge J P, Zarrinpar A. Quantitative Measure of Intestinal Permeability Using Blue Food Coloring. J Surg Res. 2019; 233:20-5. Epub 2018/07/27. doi: 10.1016/j.jss.2018.07.005. PubMed PMID: 30502249; PMCID: PMC6561122.
46. Roufosse C, Simmonds N, Clahsen-van Groningen M, Haas M, Henriksen K J, Horsfield C, Loupy A, Mengel M, Perkowska-Ptasińska A, Rabant M, Racusen L C, Solez K, Becker J U. A 2018 Reference Guide to the Banff Classification of Renal Allograft Pathology. Transplantation. 2018; 102(11):1795-814. doi: 10.1097/TP.0000000000002366. PubMed PMID: 30028786.
47. Corapi K M, Chen J L, Balk E M, Gordon C E. Bleeding complications of native kidney biopsy: a systematic review and meta-analysis. Am J Kidney Dis. 2012; 60(1):62-73. Epub 2012/04/24. doi: 10.1053/j.ajkd.2012.02.330. PubMed PMID: 22537423.
48. Sigdel T K, Archila F A, Constantin T, Prins S A, Liberto J, Damm I, Towfighi P, Navarro S, Kirkizlar E, Demko Z P, Ryan A, Sigurjonsson S, Sarwal R D, Hseish S C, Chan-On C, Zimmermann B, Billings P R, Moshkevich S, Sarwal M M. Optimizing Detection of Kidney Transplant Injury by Assessment of Donor-Derived Cell-Free DNA via Massively Multiplex PCR. J Clin Med. 2018; 8(1). Epub 2018/12/23. doi: 10.3390/jcm8010019. PubMed PMID: 30583588; PMCID: PMC6352163.
49. Zimmermann B, Hill M, Gemelos G, Demko Z, Banjevic M, Baner J, Ryan A, Sigurjonsson S, Chopra N, Dodd M, Levy B, Rabinowitz M. Noninvasive prenatal aneuploidy testing of chromosomes 13, 18, 21, X, and Y, using targeted sequencing of polymorphic loci. Prenat Diagn. 2012; 32(13):1233-41. Epub 2012/10/30. doi: 10.1002/pd.3993. PubMed PMID: 23108718; PMCID: PMC3548605.
50. Servais A, Meas-Yedid V, Toupance O, Lebranchu Y, Thierry A, Moulin B, Etienne I, Presne C, Hurault dL, Le Pogamp P, Le Meur Y, Glotz D, Hayem C, Olivo Marin J C, Thervet E. Interstitial fibrosis quantification in renal transplant recipients randomized to continue cyclosporine or convert to sirolimus. Am J Transplant. 2009; 9(11):2552-60. doi: 10.1111/j.1600-6143.2009.02803.x. PubMed PMID: 19843033.
51. Pontrelli P, Rossini M, Infante B, Stallone G, Schena A, Loverre A, Ursi M, Verrienti R, Maiorano A, Zaza G, Ranieri E, Gesualdo L, Ditonno P, Bettocchi C, Schena F P, Grandaliano G. Rapamycin inhibits PAI-1 expression and reduces interstitial fibrosis and glomerulosclerosis in chronic allograft nephropathy. Transplantation. 2008; 85(1):125-34. doi: 10.1097101.tp.0000296831.91303.9a. PubMed PMID: 18192922.
52. Rivelli R F, Gonçalves RT, Leite M, Santos M A, Delgado A G, Cardoso L R, Takiya C M. Early withdrawal of calcineurin inhibitor from a sirolimus-based immunosuppression stabilizes fibrosis and the transforming growth factor-β signalling pathway in kidney transplant. Nephrology (Carlton). 2015; 20(3):168-76. doi: 10.1111/nep.12368. PubMed PMID: 25404086.
53. Burker E J, Madan A, Evon D, Finkel J B, Mill M R. Educational level, coping, and psychological and physical aspects of quality of life in heart transplant candidates. Clin Transplant. 2009; 23(2):233-40. Epub 2009/02/05. doi: 10.1111/j.1399-0012.2009.00966.x. PubMed PMID: 19208104.
54. Ware J E, Sherbourne C D. The MOS 36-item short-form health survey (SF-36). I. Conceptual framework and item selection. Med Care. 1992; 30(6):473-83. PubMed PMID: 1593914.
55. Terada I, Hyde C. The SF-36: an instrument for measuring quality of life in ESRD patients. EDTNA ERCA J. 2002; 28(2):73-6, 83. doi: 10.1111/j.1755-6686.2002.tb00206.x. PubMed PMID: 12216848.
56. Lam W Y, Fresco P. Medication Adherence Measures: An Overview. Biomed Res Int. 2015; 2015:217047. Epub 2015/10/11. doi: 10.1155/2015/217047. PubMed PMID: 26539470; PMCID: PMC4619779.
57. Tan X, Patel I, Chang J. Review of the four item Morisky Medication Adherence Scale (MMAS-4) and eight item Morisky Medication Adherence Scale (MMAS-8). Innovations in Pharmacy. 2014; 5(3):1-8. doi: 10.24926/iip.v5i3.347.
58. Bergmann M M, Byers T, Freedman D S, Mokdad A. Validity of self-reported diagnoses leading to hospitalization: a comparison of self-reports with hospital records in a prospective study of American adults. Am J Epidemiol. 1998; 147(10):969-77. doi: 10.1093/oxfordjournals.aje.a009387. PubMed PMID: 9596475.
59. Fitzmaurice G, Davidian M, Verbeke G, Molenberghs G. Longitudinal Data Analysis: Chapman & Hall/CRC; 2008. 618 p.
60. U.S. Pat. No. 10,603,390 to Chen and Ho.
61. Golubovic B, Prostran M, Miljkovic B, Vucicevic K, Radivojevic D, Grabnar I. Population Pharmacokinetic Approach of Immunosuppressive Therapy in Kidney Transplant Patients. Curr Med Chem 2016; 23(19):1998-2011.
62. Oellerich M, Christenson R H, Beck J, Schuitz E, Sherwood K, Price C P et al. Donor-Derived Cell-Free DNA Testing in Solid Organ Transplantation: A Value Proposition. The journal of applied laboratory medicine 2020; 5(5).
63. Beck J, Bierau S, Balzer S, Andag R, Kanzow P, Schmitz J et al. Digital droplet PCR for rapid quantification of donor DNA in the circulation of transplant recipients as a potential universal biomarker of graft injury. Clinical Chemistry 2013; 59(12).
64. Oellerich M, Shipkova M, Asendorf T, Walson P D, Schauerte V, Mettenmeyer N et al. Absolute quantification of donor-derived cell-free DNA as a marker of rejection and graft injury in kidney transplantation: Results from a prospective observational study. Am J Transplant 2019; 19(11):3087-3099.
65. Filippone E J, Farber J L. The Monitoring of Donor-derived Cell-free DNA in Kidney Transplantation. Transplantation 2021; 105(3):509-516.
66. Czock D, Keller F, Rasche F M, Häussler U. Pharmacokinetics and pharmacodynamics of systemically administered glucocorticoids. Clin Pharmacokinet 2005; 44(1):61-98.

1. A method of providing immunosuppressive therapy to a subject in need of immunosuppression, comprising:
a) administering known dosages of a combination of N immunosuppressive drugs to the subject;
b) performing a time course of p measurement instances of the dosages of the N drugs in the patient;
c) performing a time course of p measurement instances of the therapeutic outcome of the patient in response to the N immunosuppressive drugs;
d) fitting results of the measurements of the dosages of the N drugs in the patients and the therapeutic outcomes of the patients to a prediction of therapeutic outcome;
e) using the model of the therapeutic outcome to identify preferred changes to the dosages of the N immunosuppressive drugs; and
f) treating the patient with the changed dosages of the N immunosuppressive drugs,
wherein N is an integer of 2 or more,
wherein the model of therapeutic outcome is a quadratic function of dosages of the N drugs with m parameters where m=1+2N+(N(N−1))/2, wherein m is the number of parameters needed, and
wherein p≥m.

2. A method of claim 1 wherein the subject is a transplant patient who has received a transplant of tissue or one or more organs.

3. A method of claim 2 wherein the tissue or organ is selected from the group consisting of bone marrow, liver, kidney, heart, lung, pancreas, intestine, skin, and a combination thereof.

4. The method of claim 1, wherein N is 3 or more.

5. The method of claim 1 wherein p=m.

6. The method of claim 1, wherein the N immunosuppressive drugs include at least one calcineurin inhibitor and at least one glucocorticoid.

7. The method of claim 1, wherein the N immunosuppressive drugs are selected from two or more of the group consisting of tacrolimus, sirolimus, everolimus, zotarolimus, cyclosporine, dactinomycin, methotrexate, prednisone, mycophenolate motetil, azathioprine, basiliximab, cyclophosphamide, mycophenolic acid, and methylprednisolone.

8. The method of claim 1, wherein p comprises immunosuppression drug dose, blood drug concentrations, donor-derived fraction of cell free DNA (dd-cfDNA %), or aspartate aminotransferase.
